(19) United States
(12) Patent Application Publication
(10) Pub. No.: US 2018/0237362 A1
(43) Pub. Date: Aug. 23, 2018

(54) NOVEL POLYGODIAL ANALOGS FOR THE TREATMENT OF CANCER AND OTHER PROLIFERATIVE DISEASES

(71) Applicants: THE BOARD OF REGENTS OF THE UNIVERSITY OF TAXAS SYSTEM, Austin, TX (US); TEXAS STATE UNIVERSITY - SAN MARCOS, San Marcos, TX (US)

(72) Inventors: Cara B. GONZALES, San Antonio, TX (US); Alexander KORNIENKO, San Marcos, TX (US); Ramesh DASARI, San Marcos, TX (US)

(21) Appl. No.: 15/752,960

(22) PCT Filed: Aug. 17, 2016
(86) PCT No.: PCT/US16/47401

§ 371 Date: Feb. 15, 2018

Related U.S. Application Data
(60) Provisional application No. 62/208,656, filed on Aug. 22, 2015.

(51) Int. Cl. C07C 13/48; A61P 35/02

(52) U.S. Cl. CPC C07C 13/48; A61P 35/02

(57) ABSTRACT

The present disclosure relates generally to derivatives of polygodial and methods of use thereof. In some aspects, the present disclosure relates to using polygodial derivatives to treat cancer or other hyperproliferative diseases.

This application claims benefit of priority to U.S. Provisional Application Ser. No. 62/208,656, filed Aug. 22, 2015, the entire contents of which are hereby incorporated by reference.

This invention was made with government support under P20GM103451 awarded by the National Institute of General Medical Sciences, under CA186046-01A1 awarded by the National Cancer Institute, and NSF 0946998 awarded by the National Science Foundation. The government has certain rights in the invention.

### BACKGROUND

### I. Field

The present disclosure relates generally to novel polygodial derivatives, their use in pharmaceutical compositions, methods of using the compounds for treating diseases.

### II. Description of Related Art

Apoptosis resistance is a hallmark of cancer, because defects in apoptosis regulators invariably accompany tumorigenesis and sustain malignant progression. Therefore, because most standard chemotherapeutic agents work by the induction of apoptosis in cancer cells, its disruption during tumor evolution can promote drug resistance and result in therapy failure (Kaufmann & Earnshaw, 2000, Komienko et al., 2013, Savage et al., 2009 and Wilson et al., 2009). Indeed, many types of cancer, such as the tumors of the lung, liver, stomach, esophagus, pancreas as well as melanomas and gliomas, are intrinsically resistant to the induction of apoptosis and thus refractory to the most of the currently available chemotherapeutic agents (Brenner, 2002). For example, patients afflicted by glioblastoma multiforme (GBM) (Agnihotri et al., 2013), which responds poorly to conventional chemotherapy with proapoptotic agents (Adamson et al., 2009, Stupp et al., 2008 and Krakstad & Chekenya, 2010), have a median survival expectancy of less than 14 months when treated with the best available protocol (Stupp et al., 2005). GBM is characterized by a deregulated tumor genome containing opportunistic deletions of tumor suppressor genes as well as amplification or mutational hyperactivation of receptor tyrosine kinase receptors. These genetic changes result in enhanced survival pathways and systematic defects in the apoptotic machinery. One solution to apoptosis resistance entails the complementation of cytotoxic therapeutic regimens with cytostatic agents and thus a search for novel cytostatic anticancer drugs that can overcome cancer cell resistance to apoptosis is an important pursuit (Van Goietsenoven et al., 2010, Lamoral-Theys et al., 2009, Evdokimov et al., 2011, Luchetti et al., 2012, Aksenov et al., 2015, Masi et al., 2015, Dasari et al., 2014, Magedov et al., 2013 and Lamoral-Theys et al., 2010).

Furthermore, tumors that initially respond to chemotherapy can still become refractory to the continuing treatment by developing a multi-drug resistant phenotype (MDR) affecting a broad spectrum of structurally and mechanistically diverse antitumor agents (Gottesman et al., 2002 and Saraswathy et al., 2013). The development of MDR is common with many conventional chemotherapies, including the well-known vinca alkaloids (Chen et al., 2000) and taxanes (Geney et al., 2002). Thus, the search for agents capable of circumventing MDR mechanisms is another important area of research aiming to combat drug-resistant cancers (Frolova et al., 2013).

### SUMMARY

In accordance with the present disclosure, there is provided compounds of the formula:

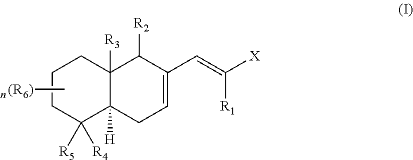

wherein:
X is an electron-withdrawing group;
R₁ is hydrogen or alkyl_{(C≤12)}, cycloalkyl_{(C≤12)}, or a substituted version of either of these groups;
R₂ is acyl_{(C≤12)} or substituted acyl_{(C≤12)};
R₃ is hydrogen, alkyl_{(C≤12)}, cycloalkyl_{(C≤12)}, substituted alkyl_{(C≤12)} or substituted cycloalkyl_{(C≤12)};
R₄ and R₅ are each independently hydrogen, alkyl_{(C≤12)}, cycloalkyl_{(C≤12)}, substituted alkyl_{(C≤12)} or substituted cycloalkyl_{(C≤12)}; or R₄ and R₅ are taken together and are alkanediyl_{(C≤8)} or substituted alkanediyl_{(C≤8)};
R₆ is amino, cyano, halo, hydroxy, or nitro; alkyl_{(C≤6)}, cycloalkyl_{(C≤6)}, acyl_{(C≤6)}, alkoxy_{(C≤6)}, acyloxy_{(C≤6)}, amido_{(C≤6)}, or a substituted version of any of these groups; and
n is 0, 1, 2, 3, or 4;
   or a pharmaceutically acceptable salt, stereoisomer, or tautomer thereof. In some embodiments, the compounds are further defined as:

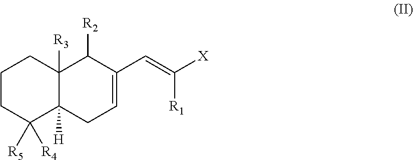

wherein:
X is an electron-withdrawing group;
R₁ is hydrogen or alkyl_{(C≤12)}, cycloalkyl_{(C≤12)}, or a substituted version of either of these groups;
R₂ is acyl_{(C≤12)} or substituted acyl_{(C≤12)};
R₃ is hydrogen, alkyl_{(C≤12)}, cycloalkyl_{(C≤12)}, substituted alkyl_{(C≤12)} or substituted cycloalkyl_{(C≤12)}; and
R₄ and R₅ are each independently hydrogen, alkyl_{(C≤12)}, cycloalkyl_{(C≤12)}, substituted alkyl_{(C≤12)} or substituted cycloalkyl_{(C≤12)}; or R₄ and R₅ are taken together and are alkanediyl_{(C≤8)} or substituted alkanediyl_{(C≤8)};
   or a pharmaceutically acceptable salt, stereoisomer, or tautomer thereof. In some embodiments, the compounds are further defined as:

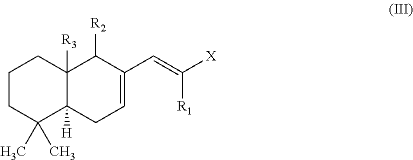

wherein:
X is an electron-withdrawing group;
R₁ is hydrogen or alkyl_{(C≤12)}, cycloalkyl_{(C≤12)}, or a substituted version of either of these groups;
R₂ is acyl_{(C≤12)} or substituted acyl_{(C≤12)}; and
R₃ is hydrogen, alkyl_{(C≤12)}, cycloalkyl_{(C≤12)}, substituted alkyl_{(C≤12)} or substituted cycloalkyl_{(C≤12)};
   or a pharmaceutically acceptable salt, stereoisomer, or tautomer thereof. In some embodiments, the compounds are further defined as:

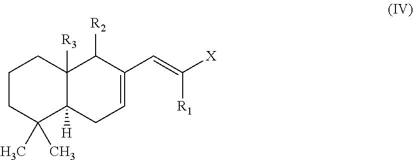

wherein:
X is an electron-withdrawing group;
R₁ is hydrogen or alkyl_{(C≤12)}, cycloalkyl_{(C≤12)}, or a substituted version of either of these groups; and
R₂ is acyl_{(C≤12)} or substituted acyl_{(C≤12)};
   or a pharmaceutically acceptable salt, stereoisomer, or tautomer thereof.

In some embodiments, the electron-withdrawing group is amino, cyano, halo, hydroxy, or nitro. In some embodiments, the electron-withdrawing group is cyano. In other embodiments, the electron-withdrawing group is acyl_{(C≤12)} or substituted acyl_{(C≤12)}. In other embodiments, the electron-withdrawing group is an alkylphosphonate_{(C≤12)}, dialkylphosphonate_{(C≤12)}, or a substituted version of either of these groups. The electron-withdrawing group may be a dialkylphosphonate_{(C≤12)} such as —P(O)(OEt)₂.

In some embodiments, the electron-withdrawing group is acetyl. In other embodiments, the electron-withdrawing group is —Y—C(O)—Z, wherein:
Y is a covalent bond, alkanediyl_{(C≤6)}, alkenediyl_{(C≤6)}, or alkynediyl_{(C≤6)}, or a substituted version of any of these groups; and
Z is hydroxy or alkoxy_{(C≤12)}, aryloxy_{(C≤12)}, aralkoxy_{(C≤12)}, or a substituted version of any of these groups.
   In some embodiments, Y is a covalent bond. In other embodiments, Y is alkenediyl_{(C≤6)} or substituted alkenediyl_{(C≤6)}. In some embodiments, Z is alkoxy_{(C≤12)} or substituted alkoxy_{(C≤12)}. In some embodiments, Z is methoxy, ethoxy, or t-butyloxy. In other embodiments, Z is aryloxy_{(C≤12)} or substituted aryloxy_{(C≤12)}. In other embodiments, Z is aralkyloxy_{(C≤12)} or substituted aralkyloxy_{(C≤12)}. In some embodiments, Z is benzyloxy. In some embodiments, R₁ is hydrogen. In other embodiments, R₁ is alkyl_{(C≤12)} or substituted alkyl_{(C≤12)}. In some embodiments, R₁ is alkyl_{(C≤6)}. In some embodiments, R₁ is methyl. In some embodiments, R₂ is acyl_{(C≤6)} or substituted acyl_{(C≤6)}. In some embodiments, R₂ is acyl_{(C≤6)}. In some embodiments, R₂ is —CHO. In some embodiments, the compound is further defined as:

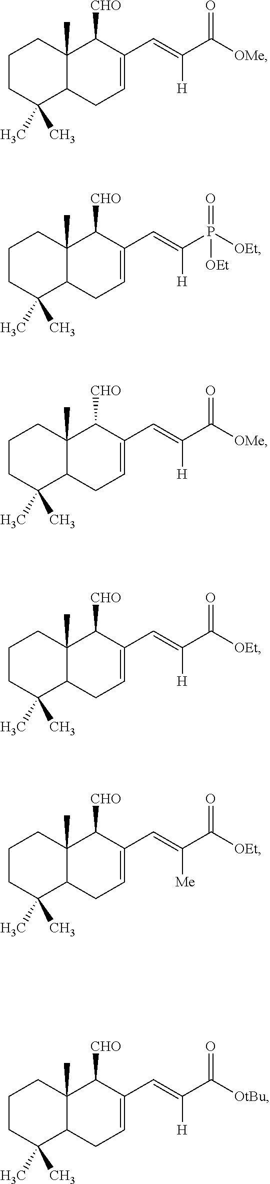

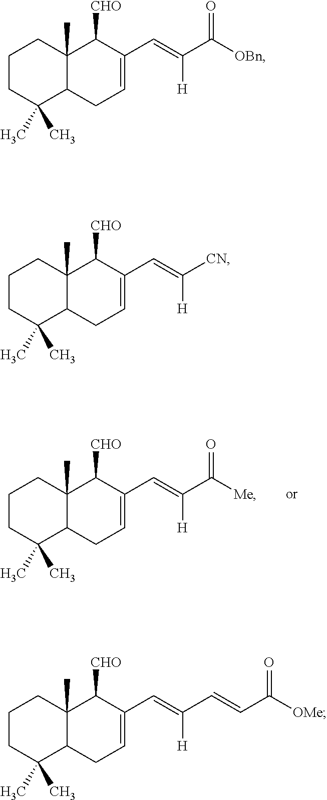

or a pharmaceutically acceptable salt, stereoisomer, or tautomer thereof.

In yet another aspect, the compound of the formula:

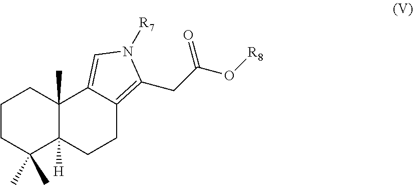

wherein:
R₇ is hydrogen or alkyl_{(C≤12)}, aralkyl_{(C≤12)}, or a substituted version of either of these groups; and
R₈ is hydrogen, alkyl_{(C≤12)}, or substituted alkyl_{(C≤12)};
   or a pharmaceutically acceptable salt, stereoisomer, or tautomer thereof. In some embodiments, R₇ is benzyl. In some embodiments, R₈ is methyl. In some embodiments, the compound is further defined as:

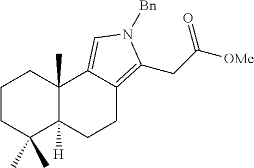

or a pharmaceutically acceptable salt, stereoisomer, or tautomer thereof.

In still yet another aspect, the present disclosure provides pharmaceutical compositions comprising:
(a) a compound of the present disclosure; and
(b) a pharmaceutically acceptable excipient.
   In some embodiments, the pharmaceutical compositions are formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crdmes, in lipid compositions, via a catheter, via a lavage, via continuous infusion, via infusion, via inhalation, via injection, via local delivery, or via localized perfusion. In some embodiments, the pharmaceutical compositions are formulated as a unit dose.

In still yet another aspect, the present disclosure provides pharmaceutical compositions comprising:
(a) polygodial, epi-polygodial, or a stereoisomer thereof; and
(b) a pharmaceutically acceptable excipient;
   formulated for administration by injection to a tumor.

In yet another aspect, the present disclosure provides methods of treating cancer in a patient comprising administering to the patient in need thereof a therapeutically effective amount of a compound or composition described herein. In some embodiments, the cancer is a carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In some embodiments, the cancer is a cancer of the lung; central nervous system; skin; breast; prostate; head, lung, neck, oral or nasal mucosa; or a solid tumor. In some embodiments, the cancer is a cancer of the head, neck, or oral or nasal mucosa. In some embodiments, the cancer is a cancer of the lung. In some embodiments, the cancer is a non-small cell lung cancer. In some embodiments, the cancer is a cancer of the central nervous system. In some embodiments, the cancer is glioblastoma or oligodendroglioma. In some embodiments, the cancer is a cancer of the breast. In some embodiments, the cancer is a cancer of the skin. In some embodiments, the cancer is melanoma. In some embodiments, the cancer is a cancer of the oral or nasal mucosa. In some embodiments, the cancer is an oral squamous cell carcinoma. In other embodiments, the cancer is a head and neck squamous cell carcinoma. In some embodiments, the cancer is a solid tumor. In some embodiments, the cancer is resistant to apoptosis. In some embodiments, the cancer is inoperable. In some embodiments, the inoperable cancer becomes operable after treatment with the compound. In some embodiments, the method comprises injecting the compound directly into the tumor. In other embodiments, the method comprises administering the compound systemically. In other embodiments, the compound is formulated for oral or intravenous administration. In some embodiments, the method further comprises administering a second therapeutic regimen to said patient. In some embodiments, the second therapeutic regimen is surgery, radiotherapy, immunotherapy, genetic therapy, or a second chemotherapeutic compound. In some embodiments, the second therapeutic regimen comprises metformin. In some embodiments, the compound reduces the tumor size such that the tumor becomes resectable.

In still yet another aspect, the present disclosure provides methods of reducing the size of a tumor comprising administering to a patient in need thereof a therapeutically effective amount of a compound or composition described herein.

In yet another aspect, the present disclosure provides methods of preparing a compound of formula I comprising reacting a compound of the formula:

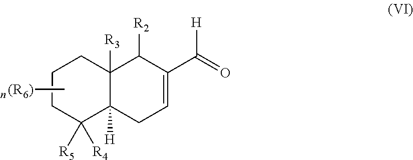

wherein:
R₂ is acyl_{(C≤12)} or substituted acyl_{(C≤12)};
R₃ is hydrogen, alkyl_{(C≤12)}, cycloalkyl_{(C≤12)}, substituted alkyl_{(C≤12)} or substituted cycloalkyl_{(C≤12)};
R₄ and R₅ are each independently hydrogen, alkyl_{(C≤12)}, cycloalkyl_{(C≤12)}, substituted alkyl_{(C≤12)} or substituted cycloalkyl_{(C≤12)}; or R₄ and R₅ are taken together and are alkanediyl_{(C≤8)} or substituted alkanediyl_{(C≤8)};
R₆ is amino, cyano, halo, hydroxy, or nitro;
alkyl_{(C≤6)}, cycloalkyl_{(C≤6)}, acyl_{(C≤6)}, alkoxy_{(C≤6)}, acyloxy_{(C≤6)}, amido_{(C≤6)}, or a substituted version of any of these groups; and
n is 0, 1, 2, 3, or 4;
   with a compound of the formula:

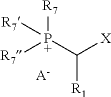

wherein:
A⁻ is a monovalent anion;
X is an electron-withdrawing group;
R₁ is hydrogen or alkyl_{(C≤12)}, cycloalkyl_{(C≤12)}, or a substituted version of either of these groups; and
R₇, R₇′, and R₇″ are each independently aryl_{(C≤12)} or substituted aryl_{(C≤12)};
   in the presence of a base. In some embodiments, the base is triethylamine.

Other objects, features and advantages of the disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula does not mean that it cannot also belong to another formula.

### BRIEF DESCRIPTION OF THE DRAWINGS

The following drawings form part of the present specification and are included to further demonstrate certain aspects of the present disclosure. The disclosure may be better understood by reference to one of these drawings in combination with the detailed description of specific embodiments presented herein.

FIG. 1—Structures of selected α,β-unsaturated 1,4-dialdehyde terpenoids.

FIGS. 2A-C. (FIG. 2A) Demonstration of the feasibility of the modified Paal-Knorr condensation of 1 to form pyrrole 2, (FIG. 2B) Paal-Knorr pyrrole formation implicated in the neurotoxicity of hexane and (FIG. 2C) novel pyrrolylation of primary amines with C12-Wittig derivatives reported herein.

FIG. 3. Synthesis of C12-Wittig derivatives 5-13 and formation of pyrrole 14 from 5 and BnNH₂.

FIGS. 4A-C. (FIG. 4A) The absence of resistant populations in all 5 cultures tested with analogue 5 and contrasting effects on viability of all cells between 5 and standard chemotherapeutic agents paclitaxel and podophyllotoxin in (FIG. 4B) A549 NSCLC and (FIG. 4C) U87 glioblastoma cell cultures. PODO=podophyllotoxin, PAO=phenyl arsine oxide.

FIGS. 5A-D. Activity of 5 against neurosphere glioma cell cultures with clinically relevant mutations. Transgenic mouse glioma cells of defined molecular subtypes were generated by forced expression of EGFRvIII (classical GBM subtype), PDGFB (proneural GBM subtype) in cdkn2a-deficient and cdkn2a/TRPV1-doubly deficient subventricular neural precursors (NPC). These primary cell cultures were treated for 24 hours either with 10 μM CBD versus a corresponding vehicle-control (containing 0.01% DMSO) or 20 μM of 5 versus vehicle control (0.02% DMSO). Cytotoxicity was measured 24 h after incubation and base-line cytotoxicity levels in the controls were arbitrarily defined as 1. Read-outs from treated cells were normalized to their respective vehicle controls and -fold change of relative cytotoxicity was calculated. Each bar represents the mean±SD from two independent experiments; statistical significance, as determined by unpaired t-tests, is indicated: *** represents p<0.001; ** p<0.005; * p=0.0257; ns not significant).

FIGS. 6A-C. (FIG. 6A) Evaluation of compound 5 in a [³H]-RTX TRPV1 displacement assay. Effects of 1 and 5 at the concentration of 10 μM on the specific binding of [³H]-RTX to the vanilloid site of TRPV1 receptor from rats spinal cord membranes. Results are expressed as mean±S.E.M from 3 independent experiments, analyzed by one way analysis of variance (ANOVA), followed by Dunnett's multiple comparison test (***p<0.05 and ****p<0.001). (FIG. 6B and FIG. 6C) Evaluation of 5 for TRPV1 activity in MDA-MB-231 breast cancer cells. (FIG. 6B) Effect of 1 (80 μM) on MDA-MB-231 [Ca²⁺]ᵢ. (FIG. 6C) Effect of 5 (20 μM) on MDA-MB-231 [Ca²⁺]ᵢ.

FIG. 7. Molecular modeling showing the capsaicin binding region of TRPV1, with the likely binding pose of capsaicin (left). Compound 1 is also well accommodated in this pocket (middle), but the Wittig derivative 5 (right—displayed in orange and overlayed with 1 in green) is required to bind with its apolar ester chain embedded in the polar “southern” region of the pocket (as highlighted by the solvent interpolated charge surface).

FIG. 8. In vitro videomicroscopic analysis of the anticancer effects of 1 and its C12-Wittig derivatives 5 and 13. The U373 human glioma cell line was treated with polygodial, 5 and 13 at their mean GI₅₀ concentrations (Table 1) or left untreated. Videomicroscopy enabled taking pictures of the culture field every 4 minutes. The experiment was conducted once in triplicate. While the morphology of cells treated with 1 was fixed over time, 5 and 13 exerted cytostatic effects on U373 cells.

FIG. 9. Viability of U373 cells by trypan blue staining. U373 cells were treated for 72 h with 1 or 13 and stained with trypan blue. The experiment was conducted once in triplicate. After having taken pictures, cells were fixed with ice-cold methanol and again stained with trypan blue as internal positive control. While cells treated with 1 were all blue-stained before methanol fixation, the 13-treated cells were still alive after 72 h of treatment.

FIGS. 10A-C. Dose response curve of the OSCC cell line HSC3 and the cervical cancer cell line HeLa treated for 48 hours with polygodial (FIG. 10A), P10, isomer, (FIG. 10B), and P3, novel analog, (FIG. 10C). Polygodial (Poly), P10, and P3 reduced cancer cell viability in vitro with P10 demonstrating the most potency followed by polygodial and P3.

FIG. 11. Cell Viability Assay of HSC3 cells treated with 60 μM polygodial, P10 (isomer), and P3 (analog) (±)N-acetyl-cysteine (10 mM NAC); ***p<0.001. Cytotoxicity was reversible by the addition of the anti-oxidant, N-acetyl-cysteine (NAC).

FIGS. 12A-B Morphological changes (20×) in HeLa cells (FIG. 12Aa-d) and HSC3 cells (FIG. 12Ba-d) with no treatment (a) or treated with Polygodial 80 μM (b), P10 4 μM (c), and P3 50 μM (d) for 1 hour. Differences in morphological changes between treatments indicate potentially unique mechanisms-of-action between polygodial and P10 (isomer), and P3 (novel analog).

FIG. 13. HeLa-derived tumors treated with 40 μg/100 μl via intra-tumor injection of Polygodial, P3 (novel analog), or vehicle control every other day for 18 days. Polygodial and P3 demonstrated significant anti-tumor effects in HeLa-derived tumors; ***p<0.001 *p<0.05. Polygodial was significantly more efficacious than P3; #p<0.05. The isomer P10 was not efficacious in vivo (data not shown).

FIGS. 14A-B. (FIG. 14A) Cell Viability Assay of Cal27 cells treated with novel analogs P3 and P27 for 72 hrs. P27 is more cytotoxic than P3 at lower concentrations. (FIG. 14B) HSC3 cells treated for 48, and 72 hrs with the novel analog P27 which shows anti-proliferative effects with an IC₅₀ of ˜10 μM.

FIG. 15. Cal27-derived tumors treated with 40 μg/100 μl via intra-tumor injection of polygodial, P27 (novel analog) or vehicle control every other day for 18 days. Polygodial elicited a notable inflammatory response on days 2-4 that returned to baseline by day 6. Both polygodial and P27 demonstrated significant anti-tumor effects against Cal27-derived xenografts; ***p<0.001 *p<0.05. No significant difference in efficacy was seen between polygodial and P27, however P27 did not elicit the inflammatory response seen with polygodial from days 2-4.

FIGS. 16A-C. Calcium imaging of CHO-TRPV1 cells (FIG. 16A), HeLa cells (FIG. 16B), and HSC3 cells (FIG. 16C) treated with polygodial (80 μM), P10 (isomer; 4 μM), and P3 (analog, 50 μM); left panels. Doses were based upon IC₅₀. Reversal of polygodial activation of TRPV1 using capsazepine (CPZ; 10 μM); right panels. Calcium imaging demonstrated that polygodial activates TRPV1 while its isomer, P10, and analog, P3, do not (FIG. 16A). This calcium influx was reversible by CPZ pre-treatment confirming specificity for TRPV1 (FIG. 16A, right panel). However polygodial may be activating other cation channels in HeLa cells as CPZ did not fully reverse calcium influx in this cell line (FIG. 16B, right panel). Notably, P10 and P3 did not induce calcium influx in HeLa cells (FIG. 16B, left panel). P3 did not induce calcium influx in the OSCC cell line, HSC3 (FIG. 16C, left panel). Both polygodial and P10 did induce calcium influx in HSC3 cells (FIG. 16C, left panel). Polygodial induced calcium influx was inhibited by CPZ in HSC3 cells indicating that only TRPV1 activation is taking place in this cell line (FIG. 16C, right panel). Without wishing to be bound by any theory, it is believed that these studies indicate that P10 and P3 cytotoxic activities against cancer cells, may be due to a second mechanism(s)-of-action that is independent of TRPV1 activation (induction of ROS and/or activation of additional ion channels such as TRPA1).

### DESCRIPTION OF ILLUSTRATIVE EMBODIMENTS

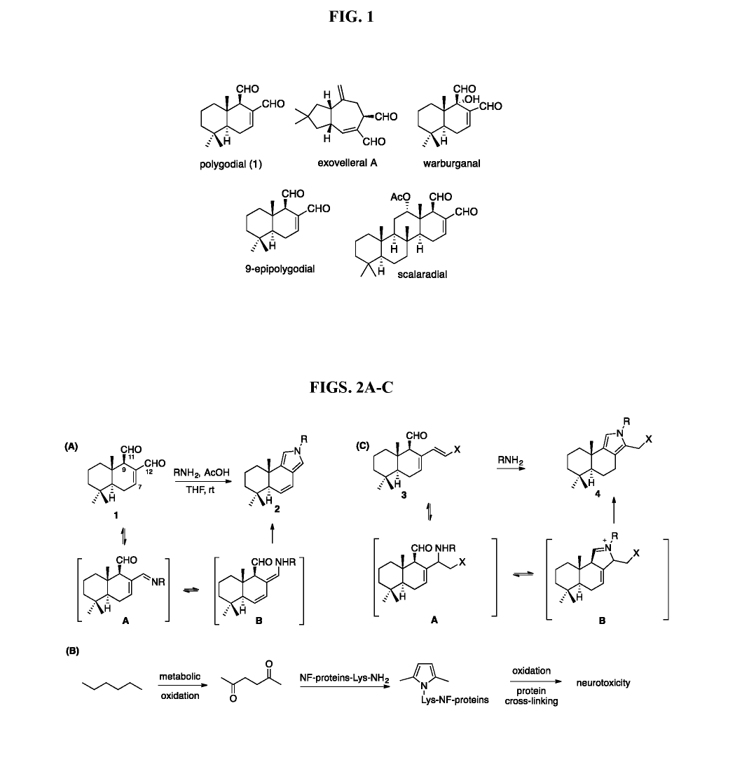

In the pursuit of agents active against drug-resistant cancers, the inventors' labs have been investigating compounds targeting ion channels (Mathieu et al., 2015 and Bury et al., 2013), whose alterations often represent important mechanisms in the impairment of apoptosis and development of drug resistance (Hoffmann & Lambert, 2014). Their attention was recently brought to the transient receptor potential vanilloid 1 receptor (TRPV1), which is a non-selective cation channel with preference for Ca²⁺ and identified as molecular target of a pungent component of hot chili pepper capsaicin, used as a spice in the culinary traditions of many countries and as an antinociceptive in traditional medicine (Caterina et al., 1997). Research has shown that in addition to its expression in sensory neurons and involvement in different modalities of pain, TRPV1 is also upregulated in various human cancer cells (Premkumar & Bishnoi, 2011, Hartel et al., 2006 and Ziglioli et al., 2009). It has thus been proposed to be a an attractive target for the treatment of brain tumors (Stock et al., 2012), as it was shown that its activation with endogenous agonists leads to endoplasmic reticulum (ER) stress in human glioma cells, followed by cell death (Stock et al., 2012). Many reports investigating TRPV1-targeting agents such as capsaicin Hartel et al., 2006, Athanasiou et al., 2007, Gonzales et al., 2014 and Skrzypski et al., 2014), resiniferatoxin (Hartel et al., 2006 and Farfariello et al., 2014), capsazepine (Athanasiou et al., 2007 and Gonzales et al., 2014), and SB366791 (Athanasiou et al., 2007), as potential anticancer agents, have appeared in the literature. Curiously, however, the group of α,β-unsaturated 1,4-dialdehyde terpenoids (FIG. 1), studied for many diverse biological properties (Sterner et al., 1999) and known for their TRPV1 agonistic activities (Sterner et al., 1999, Andre et al., 2004, Monica et al., 2007, Andre et al., 2006, D'Acunto et al., 2010 and Iwasaki et al., 2009) have not been studied as anticancer agents. Polygodial (FIG. 1) is the most well-known representative of these 1,4-dialdehydes and it was first isolated as a pungent component of the sprout of Persicaria hydropiper (L.) Delabre (Polygonaceae), a plant used as a popular condiment for sashimi in Japan (Ohsuka, 1963). Polygodial tastes hot to the human tongue and possesses antifeedant activities (Nakanishi & Kubo, 1977, Cimino et al., 1982 and Kubo & Ganjian, 1981), both of which are evidently mediated through TRPV1-targeting (Sterner et al., 1999, Andre et al., 2004, Monica et al., 2007, Andre et al., 2006, D'Acunto et al., 2010 and Iwasaki et al., 2009) possibly through the formation of a covalent complex (Sterner et al., 1999).

Encouraged by several earlier reports of cytotoxic activity associated with Polygodial (Allouche et al., 2009, Tozyo et al., 1992, Barrero et al., 1999, Anke & Sterner, 1991, Forsby et al., 1991, Andersson et al., 1993), the inventors prepared a series of its chemical derivatives and studied their TRPV1 agonistic and anticancer activities in detail. A related publication resulting from this study describes the discovery of useful anticancer activities associated with 9-epipolygodial (Dasari et al., Submitted). Herein, the inventors present a series of C12-Wittig derivatives of Polygodial that exert their antiproliferative action mainly through cytostatic effects and possess promising activities against cancer cells resistant to apoptosis as well as those with an MDR phenotype. Furthermore, these compounds undergo an unprecedented pyrrole formation with primary amines, a reaction that could be relevant in a biological environment and lead to the pyrrolylation of lysine residues in the target proteins through this previously unknown chemical mechanism.

### I. DEFINITIONS

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means=S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, for example, the formula

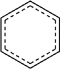

includes

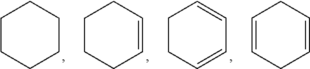

and

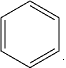

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.

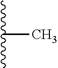

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

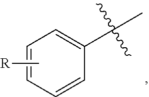

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

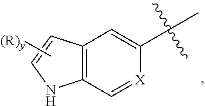

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C≤10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

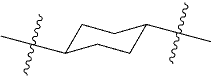 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

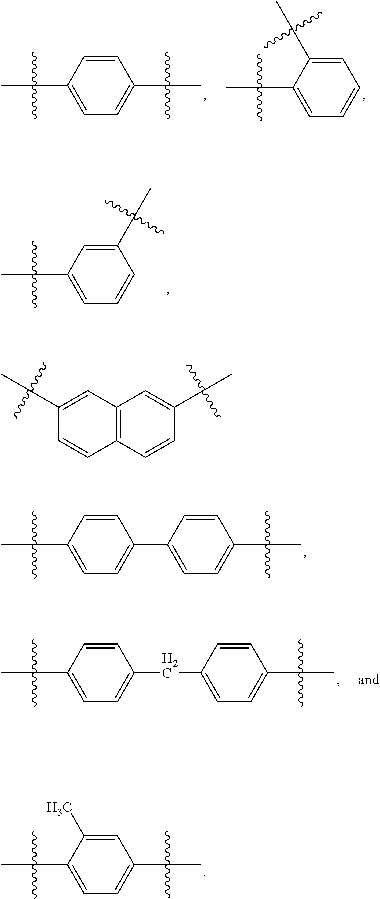

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Heteroaryl rings may contain 1, 2, 3, or 4 ring atoms selected from are nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. Heterocycloalkyl rings may contain 1, 2, 3, or 4 ring atoms selected from nitrogen, oxygen, or sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The term “alkylphosphonate” when used without the “substituted” modifier refers to the group —P(O)(OH)(OR), in which R is an alkyl, as that term is defined above. Non-limiting examples of alkylphosphonate groups include: —P(O)(OH)(OMe) and —P(O)(OH)(OEt). The term “dialkylphosphonate” when used without the “substituted” modifier refers to the group —OP(O)(OR)(OR′), in which R and R′ can be the same or different alkyl groups, or R and R′ can be taken together to represent an alkanediyl. Non-limiting examples of dialkylphosphonate groups include: —P(O)(OMe)₂, —P(O)(OEt)(OMe) and —P(O)(OEt)₂. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

The term “electron-withdrawing group” as that term is used herein, means a functional group which contains one or more atoms which contain an electronegative difference of greater than 0.4 relative to a carbon atom. Some non-limiting examples of electron-withdrawing group include nitro groups, cyano groups, carboxylic acids, phosphates, phosphonates, esters, amides, hydroxyls, and amines.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e., an enzyme, cell, cell receptor or microorganism) by half. In some embodiments, the IC₅₀ is a relative value compared to the conditions of the assay, experiment time, cells plated, or other reaction conditions.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, horse, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present disclosure which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### II. COMPOUNDS OF THE PRESENT DISCLOSURE

### A. Previously Known α,β-Unsaturated 1,4-Dialdehyde Terpenoids

The group of α,β-unsaturated 1,4-dialdehyde terpenoids (below), studied for many diverse biological properties, have only scarcely studied as anticancer agents. Polygodial is the most well-known representative of these 1,4-dialdehydes, and was first isolated as a pungent component of the sprout of Polygonum hydropiper L. (Polygonaceae), a plant used as a popular condiment for sashimi in Japan.

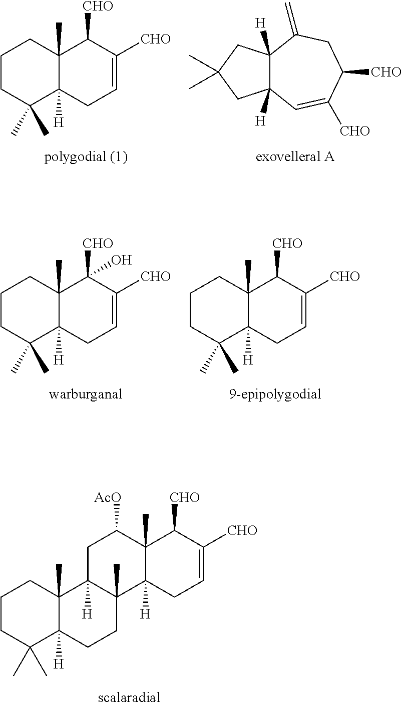

### B. Polygodial Derivatives

Encouraged by several earlier reports of cytotoxic activity associated with polygodial, the inventors have prepared a series of its chemical derivatives and studied their anti-proliferative activities in detail. Thus, the present disclosure provides a series of polygodial C12-Wittig derivatives that exert their anti-proliferative action mainly through cytostatic effects and possess promising activities against cancer cells resistant to conventional chemotherapy. These novel polygodial derivatives described in this disclosure can be prepared according to the methods described in the Examples section below. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein. In some embodiments, the present disclosure contains compounds of the formulas as described in Schemes 1 and 2.

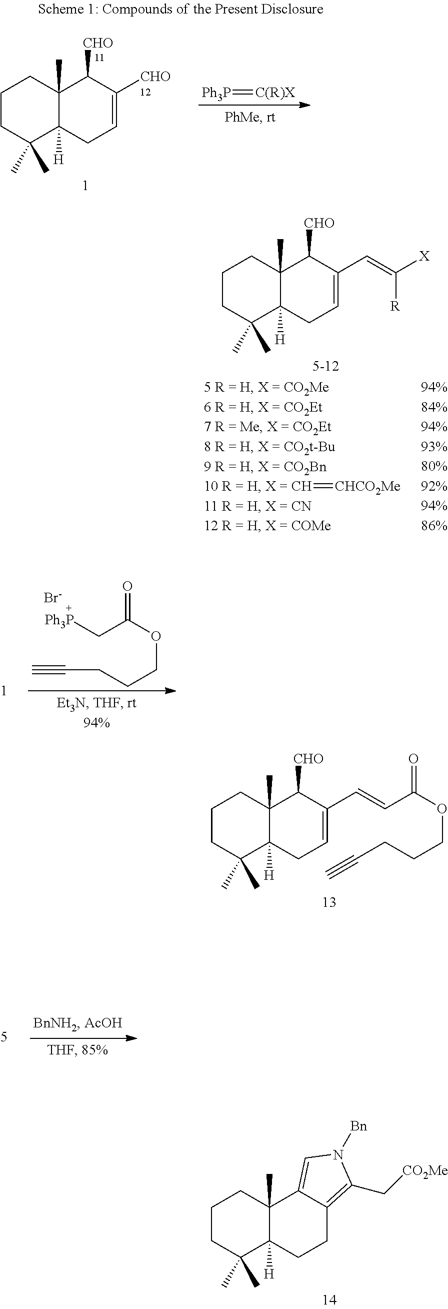

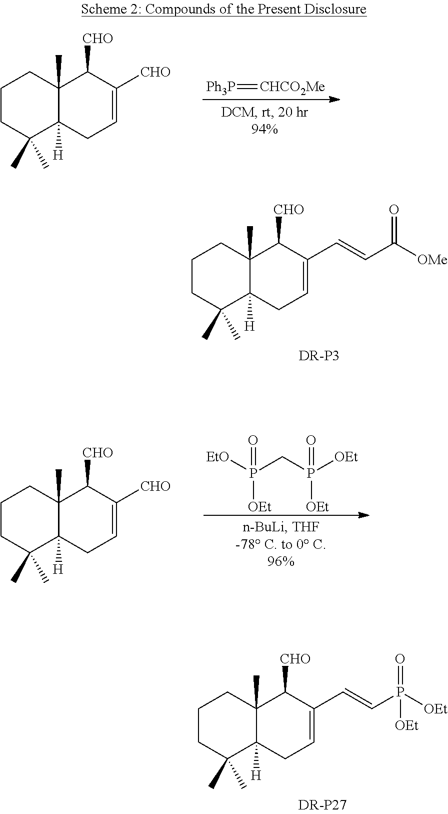

As shown above, compound 5 of Scheme 1 is also known as compound DR-P3 or simply P3. Furthermore, compound DR-P10 is also known as epi-9-polygodial, 9-epi-polygodial, or P10. Similarly, compound DR-P27 is also known as P27. The chemical studies of polygodial indicated that the C12-aldehyde is considerably more reactive than its C11-counterpart, and thus it seemed possible to prepare C12-Wittig derivatives. It was indeed discovered that polygodial reacts selectively at the C12-aldehyde functionality with a variety of stabilized phosphorous ylides, producing α,β-unsaturated esters 5-10, nitrile 11 and ketone 12 with high regioselectivity and in excellent yields. The products arising from alkenylation of the C11-aldehyde were not detected in these reaction mixtures. In addition, a terminal alkyne-containing ester 13 was prepared for a possible future conjugate synthesis through click chemistry. Finally, when 5 was reacted with BnNH₂ in THF in the presence of catalytic amount of AcOH, pyrrole 14 was cleanly formed in 85% yield.

The novel polygodial derivatives described in this disclosure may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. The novel polygodial derivatives may occur as racemates and racemic mixtures, single enantiomers, diastereomeric mixtures and individual diastereomers. In some embodiments, a single diastereomer is obtained. The chiral centers of the present disclosure can have the S or the R configuration.

In addition, atoms making up the novel polygodial derivatives of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present disclosure may be replaced by a silicon atom(s). Furthermore, it is contemplated that one or more oxygen atom(s) of the novel polygodial derivatives may be replaced by a sulfur or selenium atom(s).

The novel polygodial derivatives may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical advantages over, compounds known in the prior art for use in the indications stated herein.

Compounds of the present disclosure may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs.

Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

### III. HYPERPROLIFERATIVE DISEASES

While hyperproliferative diseases can be associated with any disease which causes a cell to begin to reproduce uncontrollably, cancer is the common example. One of the key elements of cancer is that the cell's normal apoptotic cycle is interrupted and thus agents that lead to apoptosis of the cell are important therapeutic agents for treating these diseases. In this disclosure, the novel polygodial derivatives have been shown to lead to cellular apoptosis and as such can potentially be used to treat a variety of types of cancer lines. As such, the novel polygodial derivatives may be used to effectively treat cancers such as an oral tumor such as oral squamous cell carcinoma, a tumor of the head or neck, breast cancer, cervical cancer, skin cancer, brain cancer, lung cancer, or prostate cancer. In various aspects, it is anticipated that compounds of the present disclosure may be used to treat virtually any malignancy.

Cancer cells that may be treated with the polygodial derivatives of the present disclosure according to the embodiments include but are not limited to cells from the bladder, blood, bone, bone marrow, brain, breast, colon, esophagus, gastrointestine, gum, head, kidney, liver, lung, nasopharynx, neck, oral, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### IV. PHARMACEUTICAL FORMULATIONS AND ROUTES OF ADMINISTRATION

For administration to a mammal in need of such treatment, the novel polygodial derivatives in a therapeutically effective amount are ordinarily combined with one or more excipients appropriate to the indicated route of administration. The novel polygodial derivatives may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol, and tableted or encapsulated for convenient administration. Alternatively, the novel polygodial derivatives may be dissolved in water, polyethylene glycol, propylene glycol, ethanol, corn oil, cottonseed oil, peanut oil, sesame oil, benzyl alcohol, sodium chloride, and/or various buffers. Other excipients and modes of administration are well and widely known in the pharmaceutical art.

The pharmaceutical compositions useful in the present disclosure may be subjected to conventional pharmaceutical operations such as sterilization and/or may contain conventional pharmaceutical carriers and excipients such as preservatives, stabilizers, wetting agents, emulsifiers, buffers, etc.

The novel polygodial derivatives may be administered by a variety of methods, e.g., orally or by injection (e.g., subcutaneous, intratumoral, intravenous, intraperitoneal, etc.). Depending on the route of administration, the novel polygodial derivatives may be coated in a material to protect the compound from the action of acids and other natural conditions which may inactivate the compound. They may also be administered by continuous perfusion/infusion of a disease or wound site.

To administer the therapeutic compound by other than parenteral administration, it may be necessary to coat the novel polygodial derivatives with, or co-administer the novel polygodial derivatives with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The novel polygodial derivatives may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion are also envisioned. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the novel polygodial derivatives in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The novel polygodial derivatives can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the novel polygodial derivatives may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the novel polygodial derivatives in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of the novel polygodial derivatives calculated to produce the desired therapeutic effect in association with the required pharmaceutical carrier. The specification for the dosage unit forms of the disclosure are dictated by and directly dependent on (a) the unique characteristics of the novel polygodial derivatives described in this disclosure and the particular therapeutic effect to be achieved, and (b) the limitations inherent in the art of compounding such a therapeutic compound for the treatment of a selected condition in a patient.

The therapeutic compound may also be administered topically to the skin, eye, or mucosa. Alternatively, if local delivery to the lungs is desired the therapeutic compound may be administered by inhalation in a dry-powder or aerosol formulation.

The novel polygodial derivatives describe in this disclosure are administered at a therapeutically effective dosage sufficient to treat a condition associated with a condition in a patient. For example, the efficacy of the novel polygodial derivatives can be evaluated in an animal model system that may be predictive of efficacy in treating the disease in humans, such as the model systems shown in the examples and drawings.

In some embodiments, the actual dosage amount of the novel polygodial derivatives of the present disclosure or composition comprising the novel polygodial derivatives of the present disclosure administered to a subject is determined by physical and physiological factors such as age, sex, body weight, severity of condition, the type of disease being treated, previous or concurrent therapeutic interventions, idiopathy of the subject and on the route of administration. These factors may be used by a skilled artisan to determine the appropriate dosage amount. The practitioner responsible for administration will typically determine the concentration of active ingredient(s) in a composition and appropriate dose(s) for the individual subject. The dosage may be adjusted by the individual physician in the event of any complication.

An effective amount typically will vary from about 2 mg/kg to about 50 mg/kg, in one or more dose administrations daily, for one or several days (depending of course of the mode of administration and the factors discussed above). In some particular embodiments, the amount is less than 5,000 mg per day with a range of 100 mg to 4500 mg per day.

In some embodiments, the effective amount is less than 10 mg/kg/day, less than 100 mg/kg/day, less than 250 mg/kg/day, less than 100 mg/kg/day, less than 50 mg/kg/day, less than 25 mg/kg/day or less than 10 mg/kg/day. Alternatively, in some embodiments, the range is 1 mg/kg/day to 200 mg/kg/day.

In other non-limiting examples, a dose may also comprise from about 10 mg/kg/body weight, about 100 mg/kg/body weight, about 10 g/kg/body weight, about 5 g/kg/body weight, or more per administration, and any range derivable therein. In non-limiting examples of a derivable range from the numbers listed herein, a range of about 1 mg/kg/body weight to about 100 mg/kg/body weight, about 5 g/kg/body weight to about 10 g/kg/body weight, etc., can be administered, based on the numbers described above.

In certain embodiments, a pharmaceutical composition of the present disclosure may comprise, for example, at least about 0.1% of a novel polygodial derivative described in the present disclosure. In other embodiments, the compound of the present disclosure may comprise between about 0.25% to about 75% of the weight of the unit, or between about 25% to about 60%, or between about 1% to about 10%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The novel polygodial derivatives may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the subject has eaten or will eat. In other embodiments, the disclosure is taken as a dietary supplement. In some embodiments, the novel polygodial derivatives are taken before the onset of the tumor as a prophylaxis measure. In other embodiments, the novel polygodial derivatives are taken as a treatment option for use as an antiproliferative agent.

### V. COMBINATION THERAPY

In addition to being used as a monotherapy, the novel polygodial derivatives described in the present disclosure may also find use in combination therapies. Effective combination therapy may be achieved with a single composition or pharmacological formulation that includes both agents, or with two distinct compositions or formulations, administered at the same time, wherein one composition includes a novel polygodial derivative, and the other includes the second agent(s). The other therapeutic modality may be administered before, concurrently with, or following administration of the novel polygodial derivatives. The therapy using the novel polygodial derivatives may precede or follow administration of the other agent(s) by intervals ranging from minutes to weeks. In embodiments where the other agent and the novel polygodial derivatives are administered separately, one would generally ensure that a significant period of time did not expire between the time of each delivery, such that each agent would still be able to exert an advantageously combined effect. In such instances, it is contemplated that one would typically administer the novel polygodial derivatives and the other therapeutic agent within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of a novel polygodial derivative, or the other agent will be desired. In this regard, various combinations may be employed. By way of illustration, where the novel polygodial derivatives is “A” and the other agent is “B”, the following permutations based on 3 and 4 total administrations are exemplary:
A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated. Non-limiting examples of pharmacological agents that may be used in the present disclosure include any pharmacological agent known to be of benefit in the treatment of a cancer or hyperproliferative disorder or disease. In some embodiments, combinations of the novel polygodial derivatives with a cancer targeting immunotherapy, radiotherapy, chemotherapy, or surgery are contemplated. Also contemplated is a combination of a novel polygodial derivative with more than one of the above mentioned methods including more than one type of a specific therapy. In some embodiments, it is contemplated that the immunotherapy is a monoclonal antibody which targets HER2/neu such trastuzumab (Herceptin®) or a similar antibody. In other embodiments, the immunotherapy can be other cancer targeting antibodies such as alemtuzumab (Campath®), bevacizumab (Avastin®), cetuximab (Eribitux®), and panitumumab (Vectibix®) or conjugated antibodies such as ibritumomab tiuxetan (Zevalin®), tositumomab (Bexxar®), brentuximab vedotin (Adcetris®), ado-trastuzumab emtansine (Kadcyla™), or denileukin dititox (Ontak®) as well as immune cell targeting antibodies such as ipilimumab (Yervoy®), tremelimumab, anti-PD-1, anti-4-1-BB, anti-GITR, anti-TIM3, anti-LAG-3, anti-TIGIT, anti-CTLA-4, or anti-LIGHT. Additionally, in some embodiments, the novel capsazepin derivatives can be administered with gefitinib, TAE684, tivantinib, or combinations of these drugs. Furthermore, in some embodiments, the novel polygodial derivatives are envisioned to be used in combination therapies with dendritic cell-based immunotherapies such as Sipuleucel-T (Provenge®) or adoptive T-cell immunotherapies.

Furthermore, it is contemplated that the novel polygodial derivatives are used in combination with a chemotherapeutic agent such as gefitinib, TAE684, tivantinib, anthracyclines, taxanes, methotrexate, mitoxantrone, estramustine, doxorubicin, etoposide, vinblastine, carboplatin, vinorelbine, 5-fluorouracil, cisplatin, topotecan, ifosfamide, cyclophosphamide, epirubicin, gemcitabine, vinorelbine, irinotecan, etoposide, vinblastine, pemetrexed, melphalan, capecitabine, oxaliplatin, BRAF inhibitors, and TGF-beta inhibitors. In some embodiments, the combination therapy is designed to target a cancer such as those listed above. In the preferred embodiments, the cancer the combination therapy is designed to treat is a cancer of the neck, mouth, or head, breast cancer, lung cancer, prostate cancer, cervical cancer, or other epithelial derived solid tumors.

1. Chemotherapy

In some embodiments, the polygodial derivatives of the present disclosure can be used in conjunction with one or more additional chemotherapies. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); 42inblast; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, famesyl-protein tansferase inhibitors, transplatinum, 5-fluorouracil, vincristin, vinblastin and methotrexate and pharmaceutically acceptable salts, acids or derivatives of any of the above.

2. Radiotherapy

In some embodiments, the polygodial derivatives of the present disclosure can be used in conjunction with radiotherapy. Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Without being bound by theory, radiotherapy can increase the amount of reactive oxygen species in tumor cells. In some embodiments, the combination therapy of the compounds of the present disclosure and radiotherapy can enhance the production of reactive oxygen species and thus the anti-tumor effects of the treatment. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In some embodiments, the polygodial derivatives of the present disclosure can be used in conjunction with one or more additional immunotherapies. In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-6, IL-10, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

4. Surgery

In some embodiments, the polygodial derivatives of the present disclosure can be used in conjunction with surgery. Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present disclosure to improve the anti-hyerproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

The skilled artisan is directed to “Remington's Pharmaceutical Sciences” 15^{th} Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

It also should be pointed out that any of the foregoing therapies may prove useful by themselves in treating cancer.

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Experimental Methods

General Experimental. All reagents, solvents and catalysts were purchased from commercial sources (Acros Organics and Sigma-Aldrich) and used without purification. All reactions were performed in oven-dried flasks open to the atmosphere or under nitrogen or argon and monitored by thin layer chromatography (TLC) on TLC precoated (250 m) silica gel 60 F254 glass-backed plates (EMD Chemicals Inc.). Visualization was accomplished with UV light, iodine and p-anisaldehyde stains. ¹H and ¹³C NMR spectra were recorded on a Bruker 400 spectrometer. Chemical shifts (δ) are reported in ppm relative to the TMS internal standard. Abbreviations are as follows: s (singlet), d (doublet), t (triplet), q (quartet), m (multiplet). Polygodial (1) was purchased from VWR.

General Procedure for the Wittig Reaction.

To a solution of 1 (3 mg, 0.0128 mmol, 1 eq) in dichloromethane (3 mL) was added a selected Wittig reagent (5 eq). The resultant mixture was stirred at room temperature for 20 h. After completion of the reaction, as monitored by TLC, the reaction mixture was filtered and the filtrate was concentrated under reduced pressure. The crude product was purified by preparative TLC (EtOAc/Hexane, 1:10) to obtain Wittig products (5-12).

Compound 5:

94%; ¹H NMR (400 MHz, CDCl₃) δ 9.47 (d, J=4.8 Hz, 1H), 7.33 (d, J=16.3 Hz, 1H), 6.53-6.49 (m, 1H), 5.50 (d, J=16.3 Hz, 1H), 3.71 (s, 3H), 2.83 (s, 1H), 2.37-2.16 (m, 2H), 1.87-1.80 (m, 1H), 1.53-1.44 (m, 3H), 1.38-1.30 (m, 1H), 1.23-1.16 (m, 2H), 1.00 (s, 3H), 0.94 (s, 3H), 0.90 (s, 3H); ¹³C NMR (100 MHz, C₆D₆): δ 203.8, 167.2, 146.9, 141.1, 130.7, 116.8, 62.9, 51.2, 48.3, 41.9, 40.2, 37.2, 33.2, 32.9, 24.7, 22.2, 18.3, 15.3; HRMS (ESI) calcd for C₁₈H₂₆NaO₃ (M+Na) 313.1780, found 313.1779.

Compound 6:

84%; ¹H NMR (400 MHz, C₆D₆) δ 9.36 (d, J=4.7 Hz, 1H), 7.53 (d, J=16.4 Hz, 1H), 5.89-5.86 (m, 1H), 5.87 (d, J=16.4 Hz, 1H), 4.13-4.00 (m, 2H), 2.63-2.58 (m, 1H), 1.79-1.63 (m, 3H), 1.26-1.18 (m, 3H), 1.07 (td, J=13.0, 5.8 Hz, 1H), 0.98 (t, J=8.0 Hz, 3H), 0.95-0.88 (m, 1H), 0.75-0.69 (m, 1H), 0.66 (s, 3H), 0.65 (s, 3H), 0.64 (s, 3H); ¹³C NMR (100 MHz, C₆D₆): δ 203.9, 166.8, 146.7, 140.9, 130.7, 117.3, 62.9, 60.3, 48.4, 41.9, 40.2, 37.2, 33.2, 32.9, 24.7, 22.2, 18.3, 15.3, 14.3; HRMS m/z (ESI) calcd for C₁₉H₂₈NaO₃ (M+Na) 327.1936, found 327.1933.

Compound 7:

94%; ¹H NMR (400 MHz, C₆D₆) δ 9.46 (d, J=4.4 Hz, 1H), 7.29-7.25 (m, 1H), 5.73-5.69 (m, 1H), 4.11-3.97 (m, 2H), 2.68-2.63 (m, 1H), 2.07 (d, J=1.5 Hz, 3H), 1.85-1.71 (m, 2H), 1.66-1.60 (m, 1H), 1.28-1.21 (m, 3H), 1.14-1.05 (m, 1H), 0.99 (t, J=7.1 Hz, 3H), 0.93-0.87 (m, 2H), 0.78 (s, 3H), 0.72 (s, 3H), 0.70 (s, 3H); ¹³C NMR (100 MHz, C₆D₆) δ 202.8, 167.9, 139.1, 134.0, 132.9, 130.1, 66.0, 60.7, 48.6, 42.0, 40.1, 37.2, 33.2, 32.9, 24.2, 22.0, 18.5, 15.4, 14.9, 14.3; HRMS (ESI) calcd for C₂₀H₃₀NaO₃ (M+Na) 341.2093, found 341.2091.

Compound 8:

93%; ¹H NMR (400 MHz, C₆D₆) δ 9.37 (d, J=4.7 Hz, 1H), 7.49 (d, J=16.2 Hz, 1H), 5.91-5.81 (m, 2H), 2.66-2.58 (m, 1H), 1.80-1.63 (m, 3H), 1.42 (s, 9H), 1.26-1.17 (m, 3H), 1.13-1.03 (m, 1H), 0.98-0.93 (m, 1H), 0.73 (dd, J=11.3, 5.2 Hz, 1H), 0.66 (s, 3H), 0.65 (s, 3H), 0.63 (s, 3H); ¹³C NMR (100 MHz, C₆D₆) δ 204.0, 166.3, 146.0, 140.4, 130.8, 119.0, 80.0, 62.9, 48.4, 41.9, 40.2, 37.2, 33.2, 32.9, 28.2 (3C), 24.7, 22.2, 18.3, 15.3; HRMS (ESI) calcd for C₂₁H₃₂NaO₃ (M+Na) 355.2249, found 355.2250.

Compound 9:

80%; ¹H NMR (400 MHz, C₆D₆) δ 9.32 (d, J=4.7 Hz, 1H), 7.50 (d, J=16.3 Hz, 1H), 7.24-7.20 (m, 2H), 7.11-7.03 (m, 3H), 5.86 (d, J=16.3 Hz, 1H), 5.84-5.80 (m, 1H), 5.18 (d, J=12.4 Hz, 1H), 5.06 (d, J=12.4 Hz, 1H), 2.60-2.55 (m, 1H), 1.78-1.63 (m, 3H), 1.26-1.21 (m, 3H), 1.12-1.03 (m, 1H), 0.99-0.94 (m, 1H), 0.72 (dd, J=11.3, 5.1 Hz, 1H), 0.65 (s, 6H), 0.64 (s, 3H); ¹³C NMR (100 MHz, C₆D₆) δ 203.8, 166.6, 147.3, 141.2, 136.9, 130.8, 128.7 (2C), 128.6 (2), 128.2, 116.8, 66.3, 62.8, 48.4, 41.9, 40.2, 37.2, 33.2, 32.9, 24.8, 22.2, 18.3, 15.3; HRMS (ESI) calcd for C₂₄H₃₀NaO₃ (M+Na) 389.2093, found 389.2090.

Compound 10:

92%; ¹H NMR (400 MHz, CDCl₃) δ 9.46 (d, J=5.0 Hz, 1H), 7.23 (ddd, J=15.9, 10.9, 0.8 Hz, 1H), 6.54 (d, J=15.9 Hz, 1H), 6.35-6.30 (m, 1H), 5.93 (dd, J=15.9, 10.9 Hz, 1H), 5.81 (d, J=15.9 Hz, 1H), 3.73 (s, 3H), 2.83 (s, 1H), 2.35-2.13 (m, 2H), 1.87-1.79 (m, 1H), 1.53-1.43 (m, 3H), 1.38-1.28 (m, 1H), 1.23-1.15 (m, 2H), 1.01 (s, 3H), 0.94 (s, 3H), 0.90 (s, 3H); ¹³C NMR (100 MHz, CDCl₃) δ 206.0, 167.5, 144.9, 142.5, 137.7, 131.1, 124.7, 120.3, 62.9, 51.5, 48.7, 41.8, 40.2, 37.4, 33.2, 33.1, 24.6, 22.3, 18.1, 15.6; HRMS m/z (ESI) calcd for C₂₀H₂₈NaO₃ (M+Na) 339.1936, found 339.1936.

Compound 11:

94%; ¹H NMR (400 MHz, C₆D₆) δ 9.07 (d, J=4.5 Hz, 1H), 6.37 (d, J=16.9 Hz, 1H), 5.56-5.49 (m, 1H), 4.60 (d, J=16.9 Hz, 1H), 2.29 (s, 1H), 1.72-1.51 (m, 3H), 1.26-1.15 (m, 4H), 1.09-0.87 (m, 2H), 0.63 (s, 6H), 0.57 (s, 3H); ¹³C NMR (100 MHz, C₆D₆) δ 203.0, 151.5, 141.3, 130.4, 118.0, 95.4, 62.2, 48.2, 41.8, 40.2, 37.1, 33.1, 32.8, 24.6, 22.1, 18.2, 15.3; HRMS (ESI) calcd for C₁₇H₂₃NNaO (M+Na) 280.1677, found 280.1676.

Compound 12:

86%; ¹H NMR (400 MHz, C₆D₆) δ 9.34 (d, J=4.7 Hz, 1H), 7.13 (d, J=16.5 Hz, 1H), 5.94 (d, J=16.5 Hz, 1H), 5.91-5.87 (m, 1H), 2.65-2.60 (m, 1H), 1.85 (s, 3H), 1.82-1.68 (m, 3H), 1.29-1.20 (m, 3H), 1.13 (td, J=12.8, 5.1 Hz, 1H), 1.02-0.93 (m, 1H), 0.80-0.75 (m, 1H), 0.70 (s, 3H), 0.68 (s, 3H), 0.67 (s, 3H); ¹³C NMR (100 MHz, C₆D₆) δ 203.9, 195.8, 144.2, 141.5, 130.9, 125.2, 63.0, 48.5, 41.9, 40.3, 37.3, 33.2, 32.9, 28.4, 24.9, 22.2, 18.3, 15.4; HRMS (ESI) calcd for C₁₈H₂₆NaO₂ (M+Na) 297.1830, found 297.1830.

Compound 13:

To a solution of 4-pentynyl (triphenylphosphoranylidene)acetate (11.9 mg, 0.026 mmol) in THF (2 mL) was added triethylamine (3.5 μL, 0.026 mmol) and stirred at rt for 10 min. This was followed by the addition of 1 (4.0 mg, 0.0170 mmol) in THF (1 mL) and the resultant mixture was stirred at room temperature for 20 h. After completion of the reaction, as monitored by TLC, the reaction mixture was filtered and the filtrate was concentrated under reduced pressure. The crude product was purified by preparative TLC (9/91 EtOAc/Hexane) to obtain 5.5 mg of 13 (94% yield); ¹H NMR (400 MHz, C₆D₆) δ 9.35 (d, J=4.7 Hz, 1H), 7.50 (d, J=16.3 Hz, 1H), 5.89-5.85 (m, 1H), 5.82 (d, J=16.3 Hz, 1H), 4.19-4.07 (m, 2H), 2.60 (s, 1H), 1.93 (td, J=7.1, 2.6 Hz, 2H), 1.79-1.73 (m, 1H), 1.72 (t, J=2.6 Hz, 1H), 1.71-1.60 (m, 2H), 1.58-1.49 (m, 2H), 1.27-1.18 (m, 3H), 1.14-1.04 (m, 1H), 0.99-0.89 (m, 1H), 0.73 (dd, J=11.3, 5.2 Hz, 1H), 0.67 (s, 3H), 0.66 (s, 3H), 0.65 (s, 3H); ¹³C NMR (100 MHz, C₆D₆) δ 203.9, 166.7, 147.0, 141.1, 130.7, 116.9, 83.1, 69.4, 63.1, 62.9, 48.4, 41.9, 40.3, 37.2, 33.2, 32.9, 27.9, 24.8, 22.2, 18.3, 15.4, 15.4; HRMS m/z (ESI) calcd for C₂₂H₃₀NaO₃ (M+H) 365.2093, found 365.2092.

Compound 14:

To a solution of 5 (3.4 mg, 0.012 mmol) and benzylamine (1.4 μL, 0.013 mmol) in THF (2 mL) was added AcOH (4.1 μL, 0.07 mmol). The mixture was stirred at rt for 40 h. After completion of the reaction, as monitored by TLC, the reaction mixture was concentrated under reduced pressure and co-distilled with toluene. The crude product was purified by preparative TLC (EtOAc/Hexane, 1:10) to obtain 3.8 mg of 14 (85% yield); ¹H NMR (400 MHz, C₆D₆) δ 7.09-7.03 (m, 2H), 7.02-6.98 (m, 1H), 6.91-6.86 (m, 2H), 6.21 (s, 1H), 4.86 (d, J=16.4 Hz, 1H), 4.80 (d, J=16.4 Hz, 1H), 3.31 (d, J=15.6 Hz, 1H), 3.27 (d, J=15.6 Hz, 1H), 3.15 (s, 3H), 2.80-2.72 (m, 1H), 2.60 (ddd, J=15.6, 11.8, 7.1 Hz, 1H), 2.00-1.92 (m, 1H), 1.87-1.65 (m, 3H), 1.55-1.45 (m, 2H), 1.45-1.38 (m, 2H), 1.33 (s, 3H), 1.22 (td, J=13.5, 3.7 Hz, 1H), 0.92 (s, 6H); ¹³C NMR (100 MHz, C₆D₆) δ 170.7, 139.6, 135.5, 133.0, 128.8 (2C), 128.4 (2C), 127.3, 126.7, 114.4, 52.2, 51.3, 50.7, 42.6, 40.2, 34.9, 33.8, 33.3, 30.9, 26.1, 23.2, 21.8, 20.2, 19.8; HRMS m/z (ESI) calcd for C₂₅H₃₃NNaO₂ (M+Na) 402.2409, found 402.2410.

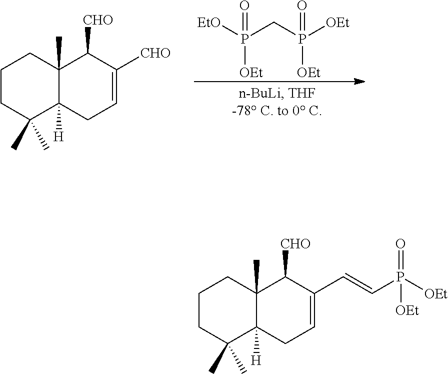

Synthesis of P27:

To a solution of tetraethyl methylenediphosphonate (36.9 mg, 0.1282 mmol) in THF (2 mL) was added n-BuLi (1.6 M) (80 μL, 0.1282 mmol) at −78° C. and stirred for 20 minutes. A solution of polygodial (10 mg, 0.04274 mmol) in THF (1 mL) was then added drop wise at −78° C. The resultant mixture was allowed to reach 0° C. and stirred for 2 hours. After completion the reaction was quenched with saturated NH₄Cl and then extracted with ethyl acetate. The organic layer was washed with water and dried over anhydrous Na₂SO₄ and then concentrated to give crude residue. The crude product was purified by prep TLC using 60% EtOAc/hexanes solvent system and obtained product in 95% yield. ¹H NMR (400 MHz, CDCl₃) δ 9.57 (d, J=5.0 Hz, 1H), 9.47 (d, J=4.8 Hz, 0.5H), 7.23-7.00 (m, 1.5H), 6.51-6.41 (m, 1.5H), 5.57 (t, J=17.4 Hz, 1H), 5.26 (t, J=17.1 Hz, 0.5H), 4.12-3.94 (m, 6H), 2.88-2.71 (m, 1.5H), 2.49 (dt, J=20.2, 5.0 Hz, 1H), 2.31 (dt, J=19.8, 4.8 Hz, 0.5H), 2.27-2.13 (m, 1.0H), 1.89-1.80 (m, 0.5H), 1.78-1.64 (m, 3H), 1.63-1.55 (m, 1.5H), 1.54-1.45 (m, 3H), 1.35-1.25 (m, 9H), 1.23-1.14 (m, 3H), 1.02-0.87 (m, 13.5H). ¹³C NMR (100 MHz, CDCl₃) δ 205.4, 201.7, 150.6, 150.5, 150.0, 149.9, 141.2, 141.2, 141.0, 140.9, 130.9, 130.7, 129.3, 129.0, 113.13, 113.1, 111.25, 111.23, 62.61, 62.60, 62.15, 62.14, 61.5 (m) 60.4, 48.6, 44.6, 42.1, 41.8, 40.2, 37.7, 37.4, 36.6, 33.2, 33.1, 32.7, 25.37, 25.36, 24.7, 24.7, 22.2, 21.9, 21.2, 21.0, 18.4, 18.0, 16.36 (m), 15.5, 14.2.

Cell Lines and Culture.

Human cancer cell lines were obtained from the American Type Culture Collection (ATCC, Manassas, Va., USA), the European Collection of Cell Culture (ECACC, Salisbury, UK) and the Deutsche Sammlung von Mikroorganismen und Zellkulturen (DSMZ, Braunschweig, Germany). The OSCC cell lines, Cal27 and HSC3 are derived from human primary tongue tumor. Cal27 cells were obtained from the American Type Culture Collection (Rockville, Md.). HSC3 cells were provided by Dr. Brian Schmidt. HeLa cells (cervical cancer cell line) were obtained from ATCC. Cells were cultured in DMEM, containing 10% FBS and 1% penicillin-streptomycin media, at 37° C. in 5% CO₂. All cells were authenticated by Genetic DNA Laboratories.

Human mammary carcinoma MCF-7 (ATCC HTB22) cells were cultured in RPMI supplemented with 10% FBS. The U87 cells (ATCC HTB-14) were cultured in DMEM culture medium, while the A549 cells (DSMZ ACC107) were cultured in RPMI culture medium supplemented with 10% heat-inactivated FBS. The GBM Hs683 (ATCC HTB-138) cells were cultivated in DMEM supplemented with 10% FBS. The human uterine sarcoma MES-SA (ATCC CRL1966) and MES-SA/D×5 cells were cultured in RPMI-1640 medium supplemented with 10% FBS with MES SA/D×5 maintained in the presence of 500 nM Doxorubicin (Sigma). SKMEL-28 cells (ATCC HTB72) and U373 GBM cells (ECACC 08061901) were cultured in RPMI culture medium supplemented with 10% heat-inactivated FBS. Cell culture media were supplemented with 4 mM glutamine (Lonza code BE17-605E), 100 μg/mL gentamicin (Lonza code 17-5182), and penicillin-streptomycin (200 units/ml and 200 μg/ml) (Lonza code 17-602E). The MDA-MB-231 (ATCC HTB-26) epithelial mammary adenocarcinoma cells were cultured in Eagle's minimum essential medium (EMEM; Invitrogen) containing 5% fetal calf serum (FCS, Cambrex), 2 mM L-glutamine (Invitrogen), 0.06% HEPES (Invitrogen) and penicillin (50 IU/ml)/streptomycin (50 lg/ml; Invitrogen) at 37° C. in a humidified atmosphere of 5% CO₂ in air. Transformed mouse NPCs were cultured in suspension under neurosphere conditions at 37° C. in a humidified atmosphere of 95% 02 and 5% CO₂ in DMEM F12 (Invitrogen 11320-074) supplemented with 1×B27 supplement (Invitrogen 17504-044), 5% penicillin-streptomycin (Biochrom 10378-017), 10 ng/ml EGF (R&D systems 236-EG), 10 ng/ml FGF (PeproTech 100-18B).

Antiproliferative Properties.

Antiproliferative properties of the synthesized compounds were evaluated by the MTT assay. All compounds were dissolved in DMSO at a concentration of either 100 μM or 50 μM prior to cell treatment. The cells were trypsinized and seeded at various cell concentrations depending on the cell type. The cells were grown for 24 h to 72 h, treated with compounds at concentrations ranging from 0.001 to 100 μM and incubated for 48 or 72 h in 100 or 200 μL media depending on the cell line used. The number of experiments and replicates varied depending on the cell line. Cells treated with 0.1% DMSO were used as a negative control; 1 μM PAO was used as a positive control.

Selection of Doxorubicin Resistant Cells.

Selection of the MES-SA/D×5 cell line was done according to Harker et al. (Harker & Sikic, 1985). The cells were split and allowed to adhere overnight. The next day cells were initially exposed to doxorubicin (DOX) at the concentration of 100 nM, which represented the GI₅₀ concentration. The cells were maintained at this DOX concentration until their growth rate reached that of the untreated cells. The DOX concentration was then increased in two-fold increments following the same growth criteria at each concentration to a final DOX concentration of 500 nM. Each new DOX concentration required approximately 2 passages to reach the growth rate of the untreated cells.

CytoTox-Fluor™ Cytotoxicity Assay.

The CytoTox-Fluor cytotoxicity assay from Promega has been used according to manufacturer's instructions. In brief, 0.015×10⁶ cells/well were plated in 24 well-plates in 450 μl (5 replicates per condition) then they received 50 μl of culture medium (DMEM-F12 without phenol red) supplemented with the drugs or respective vehicle control. After 24 hours of incubation at 37° C., 20 μl of cell suspension was transferred to a black 384 well-plate and mixed with 20 μl of bis-AAF-R₁₁₀ substrate dilution. After 2 hours of incubation at 37° C., the fluorescence intensity was measured using the Tecan InfiniteF200 fluorescence plate reader (485 nm Ex/520 nm Em). Blank was subtracted from all wells and the fluorescence read-out for untreated cells (vehicle control) was normalized to 1. Read-outs from cells receiving different treatment conditions were normalized to those of untreated cells and fold change of relative cytotoxicity compared to untreated cells was calculated for each well. Graphs were generated using the GraphPad Prism software.

[³H]-Resiniferatoxin Binding Assay.

To evaluate the possible affinity of different analogues to the vanilloid site of TRPV1, a [³H]-resiniferatoxin ([³H]-RTX) binding assay was performed as previously described (Galli et al. 2004 and Lee et al., 2006). Briefly, rats spinal cord were homogenized in buffer A (pH 7.4, 5 mM KCl, 5.8 mM NaCl, 2 mM MgCl₂, 0.75 mM CaCl₂, 137 mM sucrose, and 10 mM HEPES) and centrifuged for 10 minutes at 1000 g at 4° C. and the supernatant was further centrifuged for 30 min at 35,000 g at 4° C. The resulting pellets were than resuspended in buffer A and frozen until assayed. The binding reaction was performed in a final volume of 500 μL, containing buffer A (plus 0.25 mg/mL bovine serum albumin, BSA), membranes (0.5 mg/mL), and 2 nM [³H]-RTX in the presence or absence of analogues of 1 (10 μM). For the measurement of the nonspecific binding, 100 μM nonradioactive RTX were included used. The reaction was started by incubating tubes at 37° C. during 60 minutes, and stopped by transferring the tubes to ice bath and adding 100 μg of bovine α₁-acid glycoprotein (to reduce nonspecific binding). Finally, the bound and free membranes [³H]-RTX were separated by centrifuging for 30 min at 35,000 g at 4° C. The pellet was used to quantify the scintillation counting. The specific binding was calculated as the difference of the total and nonspecific binding and the results were measured as % of specific binding.

Intracellular Ca²⁺ measurements. Cells were grown on glass coverslips for fluorescence imaging. The cytosolic calcium was measured using Fura-2-loaded cells. Cells were loaded for 45 min at 37° C. in a humidified atmosphere of 5% CO₂ in air with 3.3 μM Fura-2/AM prepared in saline solution. Fluorescence was excited at 350 and 380 nm alternately using a monochromator (Polychrome IV; TILL Photonics, Planegg, Germany), and captured by a Cool SNAP HQ camera (Princeton Instruments, France) after filtration through a long-pass filter (510 nm). Metafluor software 7.0 (Molecular Devices) was used for acquisition and analysis. All recordings were carried out at room temperature. The cells were perfused with the saline solutions comprising of (in mM): NaCl 140, KCl 5, CaCl₂ 2, MgCl₂ 2, HEPES 10 and Glucose 5 (pH adjusted to 7.4 with NaOH).

Computer Modeling.

Molecular modelling was performed using Discovery Studio 4.5 (DS). The receptor template was obtained from the PDB (ID 3J5R) and chains B and D were retained for the simulations. Protein preparation was carried out using the Prepare Protein protocol launched from within DS. All docking simulations were carried out using a modified CDocker protocol with pregeneration of ligand conformations to adequately sample conformational space. Minimizations were carried out within DS employing the CHARMm forcefield (version 39.1).

Cell Viability.

The cytotoxicity of polygodial, P10, P3, and P27 in Cal27, HSC3, and HeLa cells was examined in vitro using a colorimetric assay, Cell Titer 96® Aqueous Non-Radioactive Cell Proliferation Assay. The GC₅₀ values were determined, after 24, 48, and/or 72 hr treatments. GraphPad Prism4 was used to perform statistical analysis.

Mouse Xenograft Models.

Six-week old female athymic nude mice were used in laminar air-flow cabinet in pathogen-free conditions. They were acclimated for one week prior to the start of the study and kept at controlled temperature and humidity, with food and water. Mice were injected subcutaneously in the flank with 2×10⁶ Cal27 cells or HeLa cells in 0.1 mL of sterile PBS. When the tumors reached 100 mm³ (Cal27) and 150 mm³ (HeLa), the mice were stratified into 4 groups, each receiving one of the following treatments every other day for two weeks: vehicle control, polygodial, P10, P3 and P27 at the concentrations noted in the figure legends.

Calcium Imaging.

CHO cells that overexpress TRPV1 (CHO-TRPV1) were treated with polygodial (80 μM), P10 (40 μM), or P3 (50 μM) and calcium influx was measured. Cells were also pre-treated with the TRPV1 antagonist capsazepine (CPZ; 10 μM) to determine if calcium influx via TRPV1 was inhibited. 3 μM ionomycin was used as a positive control. Fluo-4 Direct Calcium Assay Kit and Sweptfield confocal with Nikon Ti were used to visualize calcium influx and the images were analyzed using ImageJ software.

### Example 2—Chemistry

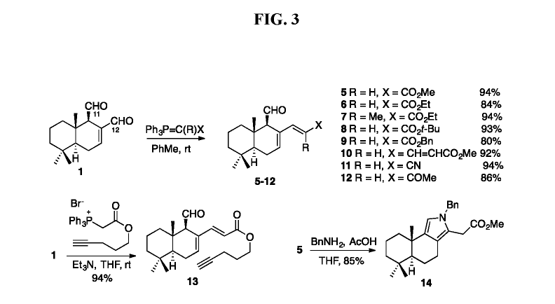

During earlier studies of reactivity of 1 toward various nucleophiles, the possibility of pyrrole formation with an active site lysine was mentioned in the literature (Cimino et al., 1982), although no pyrrole adduct was isolated in these experiments. In the inventors' attempts (Dasari et al., Submitted) to demonstrate the feasibility of what could be referred to as the modified Paal-Knorr (Amamath et al., 1991) pyrrolylation of proteins by 1, it was found that although N-alkyl pyrrole 2 (R=Bn, FIG. 2A) formed, it was extremely unstable and readily oxidized in air. This stability problem was solved by the preparation and successful isolation of electron-deficient N-aryl pyrroles 2 (R=Ph and p-NO₂—C₆H₄); however, these adducts are obviously not relevant biologically as the resultant covalent complex with a lysine residue would be an alkyl pyrrole and thus also readily oxidizable. Although examples of lysine pyrrolylation with small molecules are extremely rare (Schneimann et al., 2011), its involvement in n-hexane-induced axonal atrophy in the central nervous system is a well studied phenomenon. It has been demonstrated both in vitro and in vivo that 2,5-hexanedione, a neurotoxic n-hexane metabolite, pyrrolylates the lysine residues of axonal cytoskeleton proteins within specific regions of neurofilaments (FIG. 2B) (Zhang et al., 2010). Importantly, subsequent oxidation of the 2,5-dimethylpyrrole adducts has been shown to be an obligatory event in the induction of neuropathy (Graham et al., 1982). This line of reasoning would suggest that lysine pyrrolylation with a non-selective 1,4-dicarbonyl-containing small molecule could be generally toxic to cells, especially if the formed pyrrole adducts are unstable and may lead to further non-specific protein cross-linking events. Whether it is for this reason or another, as reported herein, the inventors' investigation of 1 as a potential anticancer agent led us to conclude that at the concentrations necessary to induce cancer cell death, 1 behaves as a toxic fixative compound with little promise as a potential drug.

It is likely that the susceptibility of N-alkyl pyrroles 2 toward oxidation is due to the presence of the conjugated C7,C8-alkene, which in addition to the electronic effects, possibly imparts significant strain by forcing the trans-fused ring B into planarity. The inventors' chemical studies of 1 indicated that the C12-aldehyde is considerably more reactive than its C11-counterpart and thus it seemed possible to prepare C12-Wittig derivatives of the type 3 in FIG. 2C, where X would be an electron-withdrawing group. Based on mechanistic considerations (intermediates A and B in FIG. 2C), the formation of pyrroles 4 appeared feasible in the reaction of 3 with primary amines and adducts 4 were predicted to be more stable due to the absence of the destabilizing C7,C8-alkene. It was indeed discovered that polygodial (1) reacts selectively at the C12-aldehyde functionality with a variety of stabilized phosphorous ylides (FIGS. 2A-C) producing α,β-unsaturated esters 5-10, nitrile 11 and ketone 12 with high regioselectivity and in excellent yields. The products arising from alkenylation of the C11-aldehyde were not detected in these reaction mixtures. In addition, a terminal alkyne-containing ester 13 was prepared for a possible future conjugate synthesis through click chemistry. Finally, when 5 was reacted with BnNH₂ in THF in the presence of catalytic amount of AcOH, pyrrole 14 was cleanly formed in 85% yield and turned out to be a stable isolable compound.

### Example 3—Results

Anticancer Evaluation.

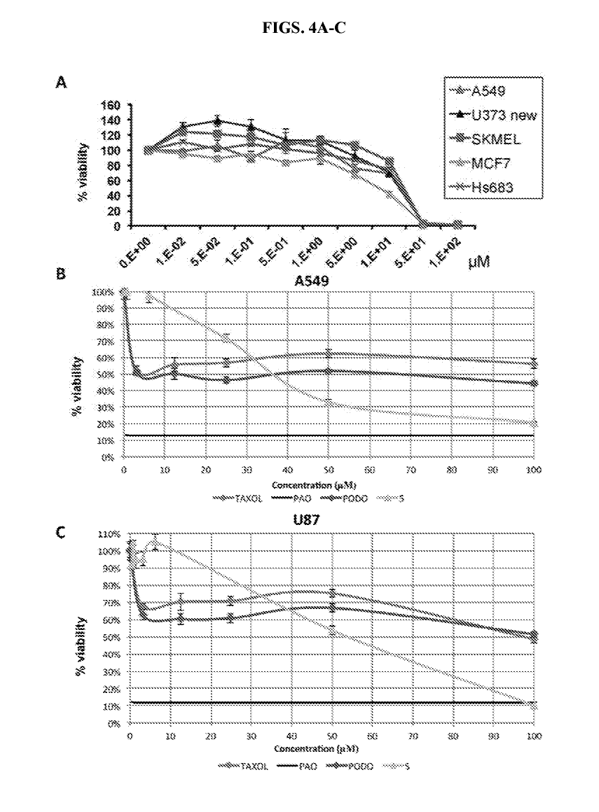

The synthesized C12-Wittig derivatives were evaluated for antiproliferative activities in a panel of cancer cell lines that included apoptosis-resistant human U373 glioblastoma (GBM) (Lefranc et al., 2013), human A549 non-small cell lung cancer (NSCLC) [61] and human SKMEL-28 melanoma (Mathieu et al., 2009) as well as apoptosis-sensitive human Hs683 anaplastic oligodendroglioma (Lefranc et al., 2013) and human MCF-7 breast cancer (Frolova et al., 2013). While 1 was ineffective in this cancer cell panel, the C12-Wittig derivatives showed moderate double-digit micromolar potencies. Of note, although the potencies in this series of compounds differed among the individual cell lines, the average GI₅₀ values over the 72-hour treatment period were quite similar, indicating either similar intracellular target binding requirements unaffected by the nature of the type of the group X (FIG. 3) or a covalent complex formation leading to an identical mode of target inhibition. Further analysis of these data revealed that the C12-Wittig derivatives did not discriminate between apoptosis resistant and apoptosis-sensitive cells and displayed comparable potencies in both cell types, indicating that compounds of this class are capable of overcoming apoptosis resistance (Table 1).

Although the moderate double-digit potencies of the synthesized derivatives are somewhat unremarkable, the analysis of the actual experimental growth curves indicated that these compounds eliminate all cells in the cultures and generate no resistant populations (close to 0% cell viability) at concentrations just slightly exceeding their GI₅₀ values, as shown for compound 5 in FIG. 4A. The contrasting effects between derivative 5 and common pro-apoptotic agents paclitaxel and podophyllotoxin on apoptosis-resistant A549 NSCLC and U87 GBM cells are shown in FIGS. 4B and 4C. Indeed, the normally low nanomolar antiproliferative agents paclitaxel and podophyllotoxin have no effect on proliferation of ca. 50% of cells at concentrations up to 100 μM (Aksenov et al., 2015), whereas 5 exhibited growth inhibitory properties against most of the cells in these cultures and, with increasing concentration, reached the antiproliferative levels of a non-discriminate cytotoxic agent phenyl arsine oxide (PAO). Compound 5 demonstrated a similar behaviour in docetaxel-resistant SCC4 and cisplatin-resistant SCC25 human oral cancer cell lines, as well as the docetaxel-resistant PC-3 human prostate cells (data not shown).

Often, tumors initially respond to chemotherapy but eventually become refractory to the continuing treatment. Such acquired resistance commonly occurs through the development of a multi-drug resistant phenotype (MDR) (Gottesman et al., 2002 and Saraswathy & Gong, 2013) affecting many common chemotherapeutic agents, including the vinca alkaloids (Chen et al., 2000) and taxanes (Geney et al., 2002). In addition, the development of apoptosis resistance and MDR mechanisms is often related and concurrent (Ruefli et al., 2002 and Johnstone et al., 1999), and thus it was of interest to evaluate 5 against MDR cells. Towards this end, the MDR uterine sarcoma cell line MES-SA/D×5, established by growing the parent uterine sarcoma MES-SA in the presence of increasing concentrations of doxorubicin and resistant to multiple functionally and structurally unrelated molecules (Harker & Sikic, 1985), was utilized. It was found that paclitaxel and vinblastine lost their potency by a factor of a thousand when tested for antiproliferative activity against the MES-SA/D×5 MDR cell line as compared with the parent MES-SA cells. In contrast, there was little variation in the sensitivities of the two cell lines towards 5 (Table 2).

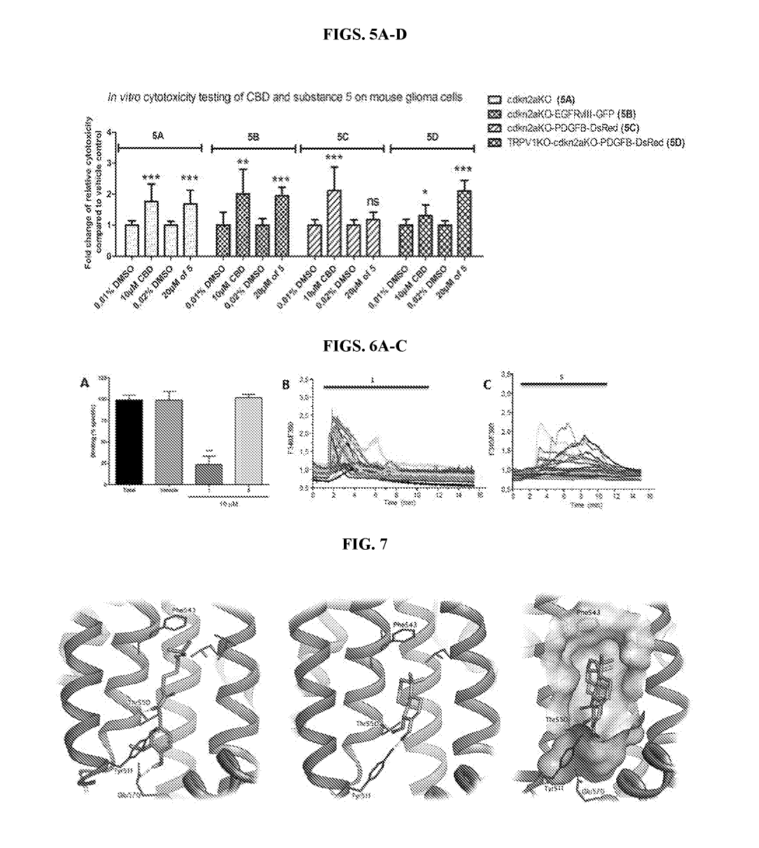

Due to the ability of 5 to overcome drug resistance, it was further challenged with a panel of GBM cell cultures maintained under neurosphere conditions. Neurospheres, known to promote the growth of stem-like cells from human glioma tissue, are generally resistant to radiation and chemotherapy (Bao et al., 2006, Liu et al., 2006, Johannessen et al., 2008 and Ma et al., 2008). Furthermore, compared with serum cultured glioma cell lines they have been shown on both histological and genetic levels to serve as a better model of human gliomas when injected into the brains of mice (Singh et al., 2004, Yuan et al., 2004, Galli et al., 2004 and Lee et al., 2006). FIGS. 5A-D shows the results of cytotoxicity evaluation for compound 5 against GBM neurosphere cultures carrying a tumor suppressor cdkn2a deletion (Purkait et al., 2013) as well as PDGFB (Guo et al., 2003) and EGFRvIII (Gan et al., 2009) and amplifications, representing frequent mutations in high-grade astrocytic tumors. The data indicate that compound 5 used at 20 μM (average GI₅₀ in Table 1) shows effectiveness similar to that of cannabidiol (CBD) at 10 μM, an orphan drug advanced to phase II clinical trials for the treatment of GBM. From the mechanistic perspective, it is interesting that CBD loses its activity against cells with the cdkn2a deletion and PDGFB amplification that additionally lack the TRPV1 gene (FIG. 5D), whereas 5 appears to be even more effective against these cells (FIG. 5B) than against their counterparts containing the intact TRPV1 (FIG. 5B). These data indicate that while the CBD-induced anticancer effects could indeed be mediated at least in part through TRPV1, which is consistent with the literature data suggesting activation of this receptor by CBD (Iannotti et al., 2014), 5 appears to work through a TURPV1-independent mechanism.

Evaluation of Vanilloid Activities.

To confirm that the synthesized derivatives of 1 do not have an effect on TRPV1, compound 5 was assessed for the inhibition of specific binding of [³H]-resiniferatoxin (RTX) in rat spinal cord membranes (Andre et al., 2006). The results (FIG. 6A) demonstrate that in contrast to 1, which displayed 76% inhibition at the concentration of 10 μM, compound 5 failed to show any activity in this assay. In a complementary assessment of the TRPV1-mediated effects, measurements of Ca²⁺ entry into MDA-MB-231 breast cancer cells abundantly expressing TRPV1 receptors (Andre et al., 2006) were performed (FIGS. 6B and C). In agreement with the [³H]-RTX TRPV1 assay, 1 at its average GI₅₀ concentration of 80 μM (from Table 1) caused a transient and synchronous [Ca²⁺]ᵢ increase in all tested cells. Moreover this Ca²⁺ response can be prevented by the TRPV1 blocker capsazepine indicating the activation of TRPV1 in the plasma membrane of MDA-MB-231 cells (data not shown). On the other hand, compound 5 at its average GI₅₀ of 20 μM (from Table 1) displayed a different pattern with sustained and asynchronous [Ca²⁺] increases displaying a different Ca²⁺-signature induced by compound 5 compared to 1.

Computer Modeling.

In an attempt to understand why the Wittig derivatization at C12 of 1 leads to compounds devoid of TRPV1 activity, capsaicin, 1 and Wittig derivative 5 were docked to the capsaicin/RTX binding site on TRPV1 (FIG. 7). The receptor for these studies was obtained by the refinement of the cryo-EM structure of TRPV1 (protein data bank (PDB) ID 3J5R) (Cao et al., 2013), as described in the inventors' related manuscript (Dasari et al., Submitted). The docking of capsaicin reveals that it is well accommodated in this pocket, with the polar phenolic moiety orientated toward the polar “southern” region of the binding pocket and the apolar alkyl chain extending up into the apolar “northern” region of the pocket (FIG. 7, left). The phenolic proton of 1 is well positioned to form a hydrogen bonding interaction to the carboxylate of Glu570 and the amide proton linking the alkyl chain is well suited to form another hydrogen bonding interaction to Thr550 (as was observed for the reduced polygodiol derivative described in the inventors' related manuscript) (Dasari et al., Submitted). Compound 1 is similarly accommodated in the pocket, although being somewhat smaller, hydrogen bonding interactions occur between the aldehydes and the residues Thr550 and Tyr511, thereby allowing the hydrophobic region of the molecule to be well placed in the “northern” hydrophobic region of the binding pocket. Interestingly, docking of the Wittig derivative 5 suggests a similar pose as the best possible orientation; however, this would require that the apolar ester chain protrudes deeply into the polar “southern” region of the pocket, with the methyl group in close proximity to the negatively charged carboxylate of Glu570. This undesirable mismatched interaction may well account for the loss of TRPV1 activity for 5.

Studies of Morphological Changes in Affected Cancer Cells.

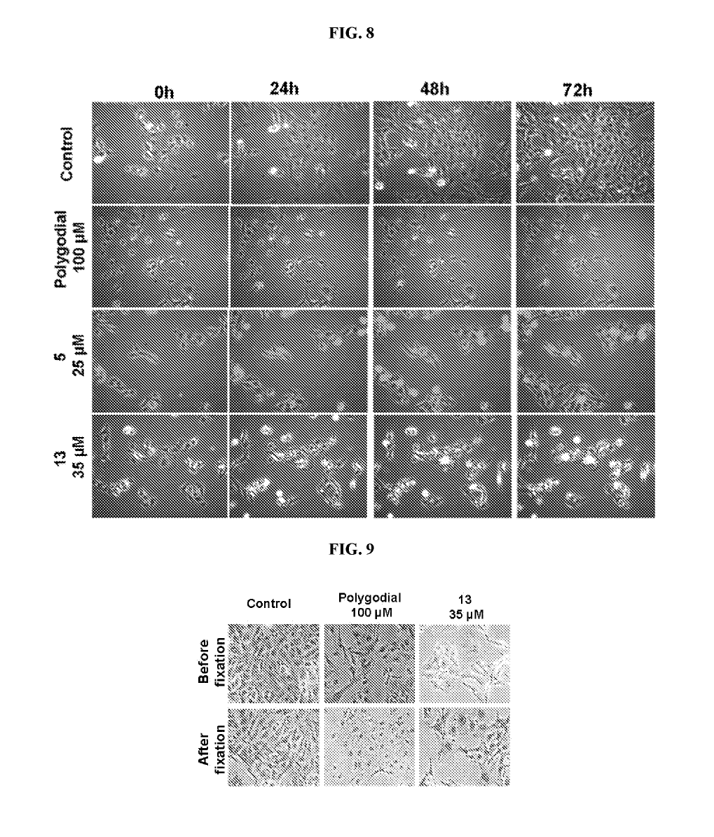

Although many reports investigating vanilloid agonists as potential anticancer agents have appeared in the literature, the involvement of TRPV1 in mediating their antiproliferative effects has been questioned on many occasions as vanilloid antagonists have consistently failed to prevent capsaicin or RTX-induced cell death (Hartel et al., 2006, Athanasiou et al., 2007, Gonzales et al., 2014, Skrzypski et al., 2014 and Farfariello et al., 2014). In the related publication (Dasari et al., Submitted), the inventors show that these observations also apply to α,β-unsaturated 1,4-dialdehyde terpenoids, such as 1 and 9-epipolygodial, and that cancer cell death induced by these compounds cannot be explained by TRPV1-targeting. In an effort to gain insight into possible mechanisms associated with antiproliferative properties of 1 and the C12-Wittig derivatives, the morphological changes in cells treated with these compounds were studied with computer-assisted phase-contrast microscopy (Luchetti et al., 2012) (quantitative videomicroscopy). FIG. 8 shows that 1, at a concentration equaling its GI₅₀ value against the U373 cell line (Table 1), exhibited strong fixative effects on these GBM cells. In contrast, the C12-Wittig derivatives 5 and 13 inhibited cancer cell proliferation without inducing cell death when assayed at their GI₅₀ concentrations (Table 1).

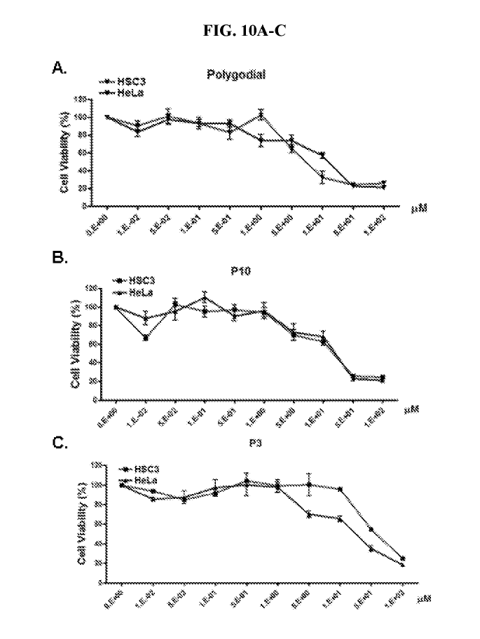

To confirm the results obtained in the videomicroscopy experiments, trypan blue assay was employed to detect necrotic and late apoptotic cells that had lost their plasma membrane integrity (FIG. 9). Indeed, while cells treated with 1 were all blue-stained before methanol fixation, the 13-treated cells were still alive after 72 hr of treatment. These observations support the results of the MTT assay and provide an explanation for the effectiveness of the C12-Wittig derivatives against cells, which display resistance to apoptosis induction. In contrast, at the concentrations necessary to induce cancer cell death 1 behaves as a toxic fixative compound of little promise as a potential drug.

Novel polygodial analogs, P3 and P27 were developed based upon the polygodial pharmacophore. The synthesis of the novel compounds P3 (DR-P3) and P27 (DR-P27) is shown in Schemes 1 and 2. A naturally-occurring isomer, P10, was also used in these studies for comparative analysis. Both P3 and P27 have significant anti-proliferative effects in vivo and in vitro (FIGS. 10-16). These effects do not appear to be tumor specific given that efficacy is shown in oral squamous cell carcinoma (OSCC) cell lines, Cal27 and HSC3, and in cervical cancer cells, HeLa, in culture and in xenografted tumor-bearing athymic nude mice.

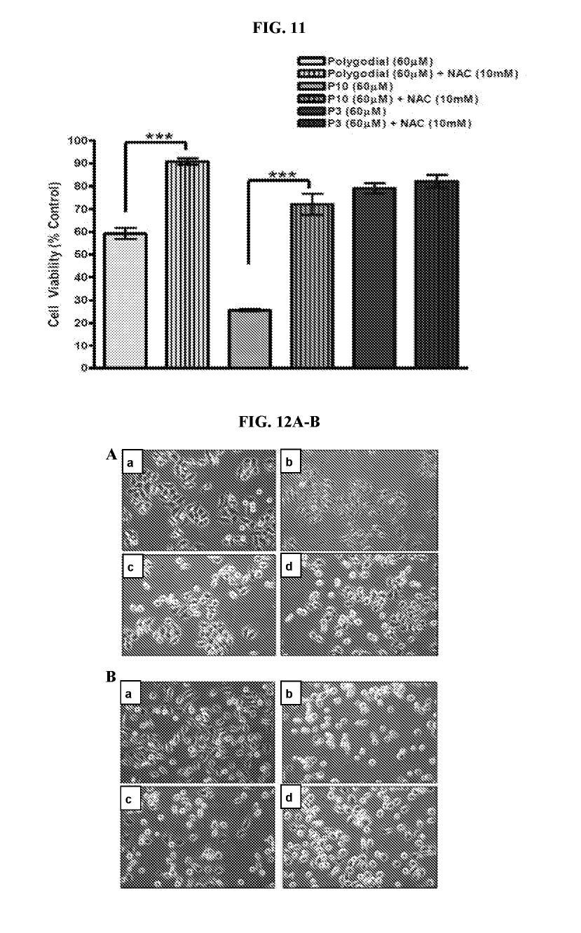

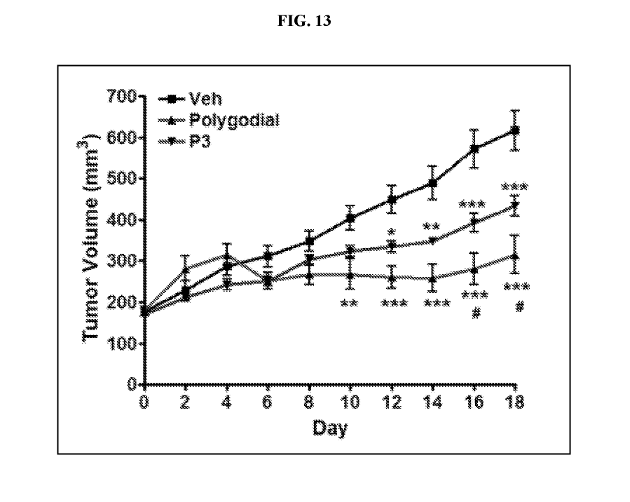

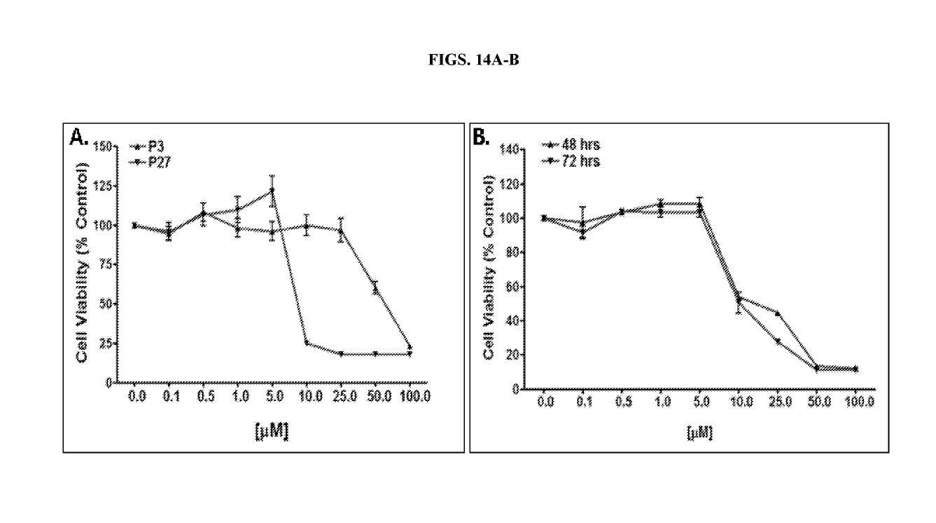

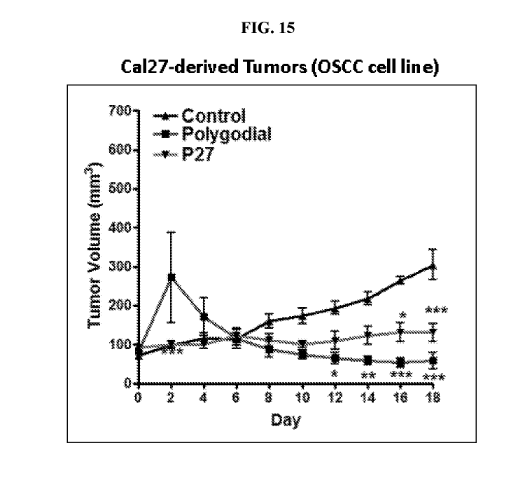

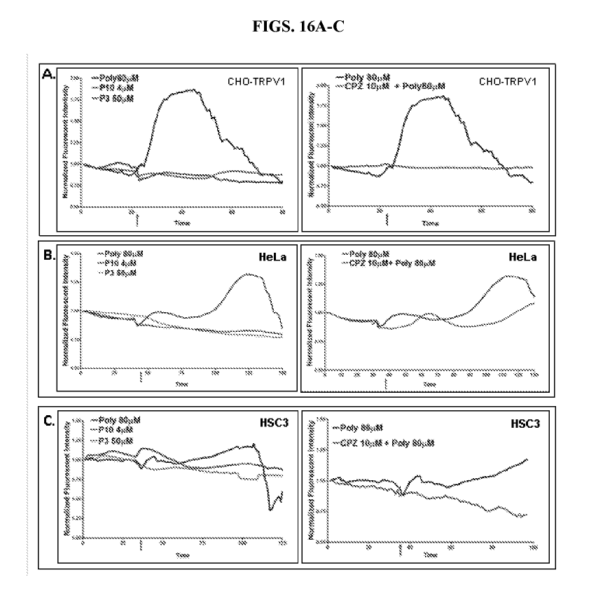

To assess the mechanism-of-action, the inventors conducted cell viability assays+/−the anti-oxidant N-Acetyl Cysteine (NAC) and calcium imaging studies. The anti-oxidant NAC reverses cytotoxicity in vitro (FIG. 11), indicating that the cytotoxic activities may be due to a second mechanism(s)-of-action that is independent of TRPV1. Calcium imaging demonstrated that Polygodial activates TRPV1 while its naturally-occurring isomer (P10) and the novel analog (P3) do not (FIG. 16A, left). Polygodial activation of TRPV1 is reversed by the TRPV1 antagonist capsazepine (CPZ; FIG. 16A, right) indicating that polygodial is specific for TRPV1. However, polygodial may be activating other cation channels in HeLa cells because subsequent calcium influx is not fully reversed by the TRPV1 specific inhibitor CPZ (FIG. 16B, right). Furthermore, while P10 (isomer) does not activate TRPV1, it may be activating other cation channels in HSC3 cells (FIG. 16C, left). The inventors demonstrated that P10 does not activate TRPV1 (FIG. 16A, left) and yet calcium influx takes place in HSC3 cells which are shown to express other calcium ion channels (FIG. 16C, left). Additionally, this calcium influx is not reversed by the TRPV1 specific inhibitor CPZ (FIG. 16C, right). Importantly, the novel analog, P3, does not induce calcium influx in any of the cell lines tested indicating a novel mechanism-of-action and eliminating potential adverse effects associated with TRPV1 activation. Taken together, these data indicate that alternate mechanism(s)-of-action which may include induction of reactive oxygen species (ROS) and/or activation of other ion channels not yet studied. Additionally, polygodial appeared to induce a marked, but transient inflammation and swelling when injected into Cal27 and HeLa xenografts of tumor bearing mice. This transient inflammation/swelling is apparent in FIGS. 13 and 15. In contrast, the novel analogs P3 and P27 and the isomer P10 showed no adverse effects in vivo, also indicative of a potential alternate mechanism-of-action. Of note, P27 was shown to be equipotent with polygodial in mouse xenograft models; however, no observable adverse effects were detected with the P27 analog. Therefore this novel analog (P27) may be efficacious for treating solid tumors in humans.

TABLE 1
In Vitro growth inhibitory effects of the C12-Wittig derivatives of 1

| | GI₅₀ in vitro values (μM)^{a} | | | | | |
|---|---|---|---|---|---|---|
| compound | A549 | SKMEL-28 | MCF-7 | U373 | Hs683 | Mean + SEM |
| 1 | 90 | 65 | 75 | >99 | 95 | >85 ± 6 |
| 5 | 21 | 27 | 7 | 22 | 24 | 20 ± 3 |
| 6 | 25 | 29 | 30 | 37 | 29 | 30 ± 2 |
| 7 | 26 | 37 | 27 | 37 | 28 | 31 ± 2 |
| 8 | 29 | 41 | 32 | 40 | 29 | 34 ± 3 |
| 9 | 28 | 39 | 27 | 39 | 31 | 33 ± 3 |
| 10 | 29 | 25 | 31 | 27 | 12 | 25 ± 3 |
| 11 | 27 | 42 | 31 | 36 | 28 | 33 ± 3 |
| 12 | 24 | 28 | 20 | 25 | 22 | 24 ± 1 |
| 13 | 30 | 36 | 30 | 35 | 33 | 33 ± 1 |
| 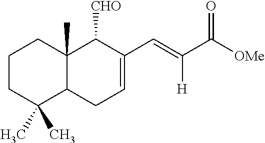 | 29 ± 1 | 70 ± 1 | 28 ± 1 | 39 ± 1 | 33 ± 0.5 | 40 ± 8 |

^{a}Average concentration required to reduce the viability of cells by 50% after a 72 h treatment relative to a control, each experiment performed in sextuplicates, as determined by MTT assay.

TABLE 2
Antiproliferative effects of 5 against MDR cells

GI₅₀ in vitro values (μM)^{a}

| | MES-SA | MES-SA/Dx5 |
|---|---|---|
| Paclitaxel | 0.007 | 10 |
| Vinblastine | 0.006 | 5 |
| 5 | 40 | 45 |

^{a}Concentration required to reduce the viability of cells by 50% after a 48 h treatment with the indicated compounds relative to a DMSO control from two independent experiments, each performed in 4 replicates, as determined by the MTT assay.

### Example 4—Conclusion

Cancers characterized by an intrinsic resistance to the induction of apoptosis are refractory to the most of the currently available chemotherapeutic agents and present a formidable clinical challenge (Kaufmann & Earnshaw, 2000, Komienko et al., 2013, Savage et al., 2009, Wilson et al., 2009 and Brenner, 2002). For example, GBM is one of the most feared of all human diseases both due to the certain fatal outcome and a rapid debilitating loss of cognitive function. The therapy-related improvement of overall survival has been counted in months, not years, for the last 40 years and the GBM clinic is in dire need of conceptually new treatment strategies (Agnihotri et al., 2013, Adamson et al., 2009 and Stupp et al., 2008). A great deal of recent research has been aimed at overcoming the apoptosis resistance of GBM cells by rendering them more susceptible to therapy-induced apoptosis (Krakstad & Chekenya, 2010). Thus, in GBM cells, several key regulatory elements of cell homeostasis and apoptosis are altered through inactivating mutations, methylation, or altered expression. These alterations affect the p53 protein, the BCL-2 protein family, the inhibitor of apoptosis proteins (IAPs) or receptor tyrosine kinases (e.g., the epidermal growth factor receptor (EGFR)) and their downstream signaling cascades. All of these represent attractive targets for therapeutic interventions and have been pursued by various researchers (Krakstad & Chekenya, 2010 and Eisele & Weller, 2013). However, to date, the results from the clinical trials of a number of such agents, mostly targeting growth factor pathways, have been disappointing. For example, a randomized, controlled, phase II trial conducted with erlotinib, a small molecule targeting the EGFR signaling, unfortunately showed no therapeutic benefit (van den Bent et al., 2009).

The identification of agents whose mode of action is not based on cell death represents an alternative approach. Indeed, delaying proliferation in cancer cells over certain periods of time will induce cell death that usually occurs via apoptosis. In this case, apoptosis is a consequence and not a cause of the drug-induced effects. The present investigation led to the discovery of such agents, which represent novel derivatives of polygodial, a sesquiterpenoid widely studied as a TRPV1 agonist. Indeed, the biological effects of these compounds are not TRPV1-mediated and compared to the parent polygodial, which displays a fixative general cytotoxic action against human cells, the C12-Wittig derivatives exert their antiproliferative action mainly through cytostatic effects explaining their activity against apoptosis-resistant cancer cells. Furthermore, these novel derivatives maintain activity against MDR cells as well as GBM neurosphere cultures carrying tumor suppressor and growth factor receptor mutations representing an import challenge in the clinical management of high-grade astrocytic tumors. These compounds are produced in an efficient one-step synthesis from polygodial using a selective Wittig derivatization of the C12-aldehyde group. Lastly, the possibility for an intriguing covalent modification of proteins through a novel pyrrole formation reaction make the described polygodial-derived chemical scaffold an interesting chemotype for the investigation of novel ways of covalent modification of proteins with small molecules of biological and therapeutic relevance.

### Example 5—Oral Cancer Studies

The inventors have developed novel compounds (P3 and P27) based upon the polygodial pharmacophore for treating solid tumors. These cancers include, but are not limited to, head and neck cancers and cervical cancer. A naturally occurring isomer, P10, was also used in these studies for comparative analysis. Both P3 and P27 have significant anti-proliferative effects in vivo and in vitro (FIGS. 10-16). These effects don't appear to be tumor specific given that efficacy is shown in oral squamous cell carcinoma (OSCC) cell lines, Cal27 and HSC3, and in cervical cancer cells, HeLa, in vitro and in xenografted tumor-bearing athymic nude mice (FIGS. 13 and 16).

Cell proliferation assays using cancer cell lines (HSC3 and HeLa) (FIGS. 10A-C), demonstrate that treatment with novel polygodial analog P3 induces dramatic reduction in cell proliferation following 48 hour treatments. The polygodial (polygodial), P3 (also described as compound 5), and P10 (epi-polygodial) are used herein.

Treatment with the antioxidant N-acetyl-L-cysteine reverses these cytotoxic effects indicating that these analogs may be causing cell death by the production of reactive oxygen species (ROS) (FIG. 11). Morphological changes seen in HSC3 and HeLa cells treated with P3 reveal apoptotic figures with as little as 1 hour of treatment (FIGS. 12Ad and 12Bd).

In vivo testing was performed in HeLa-derived xenografts generated in athymic nude mice inoculated. The HeLa-derived tumors were treated with 60 μg of analog P3 or 80 μg of polygodial every other day for 18 days. P3 treatments significantly reduced tumor growth (p<0.001) (FIG. 13). No observable adverse effects on non-malignant adjacent tissue were seen with P3 treatments; no erythema, swelling, or ulceration. Mice in all groups maintained their body weight and they resumed normal motor function following each treatment and throughout the 18 day test period. Polygodial elicited a marked inflammatory response from Day 2 until Day 6, most likely due to TRPV1 activation. Statistically, P3 was not quite as potent as polygodial, but with the advantage of no observable adverse effects, most likely because P3 fails to induce TRPV1 and subsequent chemical damage.

The novel analog, P27, showed greater potency than the P3 analog against the OSCC cell line (comparable to polygodial), Cal27, with an IC₅₀ of approximately 10 μM (FIG. 14A. Anti-proliferative effects of P27 were also confirmed against the OSCC cell line, HSC3, in vitro (FIG. 14B). The anti-tumor effects of P27 were confirmed in Cal27-derived xenografts in athymic nude mice (FIG. 15). P27 dramatically halted tumor growth throughout the experimental period yielding a significant reduction in tumor volume (p<0.001). Similarly, polygodial significantly halted tumor growth (p<0.001); however a dramatic inflammatory response indicative of TRPV1 activation was noted on Day 2 until Day 6. Conversely, P27 was significantly efficacious with equal potency of polygodial and the advantage of no observable adverse effects; no swelling, erythema or ulceration. Mice in all groups maintained their body weight and resumed normal motor function following each treatment and throughout the 18 day test period.

Scientists have noted that TRP channels are over-expressed in a number of cancer types. The inventors looked at the expression of TRPV1 in oral and cervical cancer cell lines and demonstrate P3 anti-proliferative effects in the absence of TRPV1 activation using calcium imaging (FIGS. 10, 14, and 16). Calcium imaging demonstrated that polygodial activates TRPV1 while its naturally occurring isomer (P10) and the novel analog (P3) do not (FIG. 16A, left panel). Polygodial activation of TRPV1 is reversed by the TRPV1 antagonist capsazepine (CPZ; FIG. 16A, right panel) indicating that polygodial is specific for TRPV1. However polygodial may be activating other cation channels in HeLa cells because subsequent calcium influx is not fully reversed by the TRPV1 specific inhibitor CPZ (FIG. 16B, right panel). Conversely, polygodial-induced calcium influx is fully reversed by CPZ in HSC3 cells indicating TRPV1 activity in this cell line (FIG. 16C, right panel). While P10 (isomer) does not activate TRPV1, it may be activating other cation channels in HSC3 cells. We demonstrate that P10 does not activate TRPV1 (FIG. 16A, left panel) and yet calcium influx takes place in HSC3 cells which are shown to express other calcium ion channels (FIG. 16C, left panel). Importantly, the novel analog, P3, does not induce calcium influx in any of the cell lines tested indicating a novel mechanism-of-action. Taken together, these data indicate that alternate mechanism(s)-of-action which may include induction of reactive oxygen species (ROS) and/or activation of other ion channels not yet studied. Additionally, polygodial appeared to induce a marked, but transient inflammation and swelling when injected into Cal27 and HeLa xenografts of tumor bearing mice. This transient inflammation/swelling is apparent in FIGS. 13 and 15. In contrast, the novel analogs P3 and P27 and the isomer P10 showed no adverse effects in vivo, also indicative of a potential alternate mechanism-of-action. Of note, P27 was shown to be equipotent with polygodial in mouse xenograft models; however no observable adverse effects were detected with the P27 analog. Therefore this novel analog (P27) may be efficacious for treating solid tumors in humans.

With these findings in mind and without wishing to be bound by any theory, it is believed that polygodial, P10 (isomer), P3 (novel analog), and P27 (novel analog) exerted their anti-proliferative effect in a TRPV1 independent manner. Based upon the data presented herein and without wishing to be bound by any theory, it is believed that the mechanism-of-action is most likely the production of reactive oxygen species resulting in apoptosis.

All of the compounds, formulations, and methods disclosed and claimed herein can be made and executed without undue experimentation in light of the present disclosure. While the compounds, formulations, and methods of this disclosure have been described in terms of preferred embodiments, it will be apparent to those of skill in the art that variations may be applied to the compounds, formulations, and methods, as well as in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references to the extent that they provide exemplary procedural or other details supplementary to those set forth herein, are specifically incorporated herein by reference.
Adamson et al., Expert Opin. Invest. Drugs., 18, 1061-1083, 2009.
Agnihotri et al., Arch. Immunol. Ther. Exp., 61, 25-41, 2013.
Aksenov et al., J. Med. Chem., 58, 2206-2220, 2015.
Allouche et al., Phytochemistry, 70, 546-553, 2009.
Amamath et al., J. Org. Chem., 56, 6924-6931, 1991.
Andersson et al., Toxicol In Vitro., 7, 1-6, 1993.
Andre et al., Biochem. Pharmacol., 71, 1248-1254, 2006.
Andre et al., Neuropharmacology, 46, 590-597, 2004.
Anke & Sterner, Planta Med., 57, 344-346, 1991.
Athanasiou et al., Biochem. Biophys. Res. Commun., 354, 50-55, 2007.
Austin-Ward and Villaseca, Rev. Med. Chil., 126(7):838-45, 1998.
Bao et al., Nature, 444, 756-760, 2006.
Barrero et al., J. Nat. Prod., 62, 1488-1491, 1999.
Brenner, Lancet, 360, 1131-1135, 2002.
Bukowski et al., Clin. Cancer Res., 4(10):2337-47, 1998.
Bury et al., Cell Death Dis., 4, 48-57, 2013.
Cao et al., Nature, 504, 113-118, 2013.
Caterin et al., Nature, 389, 816-824, 1997.
Chen et al., Br. J. Cancer., 83, 892-898, 2000.
Christodoulides et al., Microbiology, 144(Pt 11):3027-37, 1998.
Cimino et al., Comp. Eiorhem. Pbysiol., 73B, 471-474, 1982.
Cimino et al., Tetrahedron Lett., 25, 4151-4152, 1984.
D'Acunto et al., Tetrahedron, 66, 9785-9789, 2010.
Dasari et al., A. Synthetic and Biological Studies of sesquiterpene polygodial: activity of 9-epipolygodial against drug resistant cancer cells. Submitted
Dasari et al., Bioorg. Med. Chem Lett., 24, 923-927, 2014
Davidson et al., J. Immunother., 21(5):389-98, 1998.
Eisele & Weller, Cancer Lett., 332, 335-345, 2013.
Evdokimov et al., Bioorg. Med. Chem., 19, 7252-7261, 2011.
Farfariello et al., Chem. Biolog. Interact., 224, 128-135, 2014.
Forsby et al., Toxicol In Vitro., 5, 9-14, 1991.
Frolova et al., J. Med. Chem., 56, 6886-6900, 2013.
Galli et al. Cancer Res., 64, 7011-7021, 2004.
Gan et al., J. Clin. Neurosci., 16, 748-754, 2009.
Geney et al., Clin. Chem. Lab. Med., 40, 918-925, 2002.
Gonzales et al., Oral Oncol., 50, 437-447, 2014.
Gottesman et al., Nat. Rev. Cancer, 2, 48-58, 2002.
Graham et al., Toxicol. Appl. Pharmacol., 64, 415-422, 1982.
Guo et al., Amer. J. Pathol., 162, 1083-1093, 2003.
Handbook of Pharmaceutical Salts: Properties, and Use, Stahl and Wermuth Eds.), Verlag Helvetica Chimica Acta, 2002.
Hanibuchi et al., Int. J. Cancer, 78(4):480-485, 1998.
Harker & Sikic, Cancer Research, 45, 4091-4096, 1985.
Hartel et al., Gut, 55, 519-528, 2006.
Hellstrand et al., Acta Oncol., 37(4):347-353, 1998.
Hoffmann & Lambert, Phil. Trans. R. Soc. B., 369, 1-16, 2014.
Hui and Hashimoto, Infect. Immun., 66(11):5329-36, 1998.
Iannotti et al., ACS Chem. Neurosci., 5, 1131-1141, 2014.
Iwasaki et al., Life Sci., 85, 60-69.
Johannessen et al., Cancer Treat Rev., 34, 558-567, 2008.
Johnstone et al., Blood, 93, 1075-1085, 1999.
Ju et al., Gene Ther., 7(19):1672-1679, 2000.
Kaufmann & Earnshaw, Exp. Cell Res., 256, 42-49, 2000.
Kornienko et al., J. Med. Chem., 56, 4823-4839, 2013.
Krakstad & Chekenya, Mol. Cancer., 9, 135, 2010.
Kubo & Ganjian, Experientia 37, 1063-1064, 1981.
Lamoral-Theys et al., J. Med. Chem., 52, 6244-6256, 2009.
Lamoral-Theys, Bioorg. Med. Chem., 18, 3823-3833, 2010.
Lee et al., Cancer Cell, 9, 391-403, 2006.
Lefranc et al., J. Nat. Prod., 76, 1541-1547, 2013.
Ligresti et al., J. Pharmacol. Exp. Ther., 318, 1375-1387, 2006.
Liu et al., Mol. Cancer, 5, 67, 2006.
Luchetti et al., Chem. Med. Chem., 7, 815-822, 2012.
Ma et al., Oncogene, 27, 1749-1758, 2008.
Magedov et al., Bioorg. Med. Chem. Lett., 23, 3277-3282.
March's Advanced Organic Chemistry: Reactions, Mechanisms, and Structure, 2007.
Masi et al., Fitoterapia., 102, 41-48, 2015.
Mathieu et al., Cancer, 101, 1908-1918, 2004.
Mathieu et al., Cell. Mol. Life Sci., 2015 in press.
Mathieu et al., J Cell. Mol. Med., 13, 3960-3972, 2009.
Mitchell et al., Ann. NY Acad. Sci., 690:153-166, 1993.
Mitchell et al., J. Clin. Oncol., 8(5):856-869, 1990.
Monica et al., Bioorg. Med. Chem. Lett., 17, 6444-6447, 2007.
Morton et al., Arch. Surg., 127:392-399, 1992.
Nakanishi & Kubo, Israel J. Chem., 16, 28-31, 1977.
Ohsuka, Polygonum Hydropiper L. Nippon Kagaku Zasshi, 84, 748-752, 1963.
Pietras et al., Oncogene, 17(17):2235-49, 1998.
Premkumar & Bishnoi, M Curr. Top. Med. Chem., 11, 2192-2209, 2011.
Purkait et al., Neuropathol., 33, 405-412, 2013.
Qin et al., Proc. Natl. Acad. Sci. USA, 95(24):14411-14416, 1998.
Ravindranath and Morton, Intern. Rev. Immunol., 7: 303-329, 1991.
Rosenberg et al., Ann. Surg. 210(4):474-548, 1989.
Rosenberg et al., N. Engl. J. Med., 319:1676, 1988.
Ruefli et al., Cell Death Differ., 9, 1266-1272, 2002.
Saraswathy & Gong, Biotechnol. Adv., 31, 1397-1407, 2013.
Savage et al., Nat. Clin. Pract. Oncol., 6, 43-52, 2009.
Schneimann et al, J. Am. Chem. Soc., 133, 17494-17503, 2011.
Singh et al., Nature, 432, 396-401, 2004.
Skrzypski et al., Cell. Signal, 26, 41-48, 2014.
Sterner & Szallasi, TiPS, 20, 459-465, 1999.
Stock et al., Nat Med., 18, 1232-1238, 2012.
Stupp et al., Ann. Oncol., 19, 209-216, 2008.
Stupp et al., N. Engl. J. Med., 352, 987-996, 2005.
Tozyo et al., J. Chem. Soc. Perkin Trans. 1, 46, 1859-1866, 1992.
U.S. Pat. No. 5,739,169
U.S. Pat. No. 5,801,005
U.S. Pat. No. 5,824,311
U.S. Pat. No. 5,830,880
U.S. Pat. No. 5,846,945
van den Bent et al., J. Clin. Oncol., 27, 1268-1274, 2009.
Van Goietsenoven et al., J. Nat. Prod., 73, 1223-1227, 2010.
Wilson et al., Curr. Cancer Drug Targets, 9, 307-319, 2009.
Yuan et al., Oncogene, 23, 9392-9400, 2004.
Zhang et al., Toxicol. Sci., 117, 180-189, 2010.
Ziglioli et al., Acta. Biomed., 80, 13-20, 2009.

1. A compound of the formula: 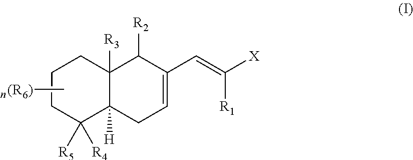
wherein:
X is an electron-withdrawing group;
R₁ is hydrogen or alkyl_{(C≤12)}, cycloalkyl_{(C≤12)}, or a substituted version of either of these groups;
R₂ is acyl_{(C≤12)} or substituted acyl_{(C≤12)};
R₃ is hydrogen, alkyl_{(C≤12)}, cycloalkyl_{(C≤12)}, substituted alkyl_{(C≤12)} or substituted cycloalkyl_{(C≤12)};
R₄ and R₅ are each independently hydrogen, alkyl_{(C≤12)}, cycloalkyl_{(C≤12)}, substituted alkyl_{(C≤12)} or substituted cycloalkyl_{(C≤12)}; or R₄ and R₅ are taken together and are alkanediyl_{(C≤8)} or substituted alkanediyl_{(C≤8)};
R₆ is amino, cyano, halo, hydroxy, or nitro;
alkyl_{(C≤6)}, cycloalkyl_{(C≤6)}, acyl_{(C≤6)}, alkoxy_{(C≤6)}, acyloxy_{(C≤6)}, amido_{(C≤6)}, or a substituted version of any of these groups; and
n is 0, 1, 2, 3, or 4;
or a pharmaceutically acceptable salt, stereoisomer, or tautomer thereof.

2. The compound of claim 1 further defined as: 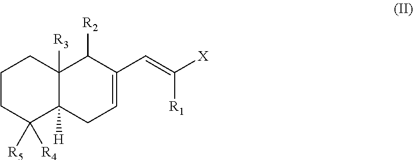
wherein:
X is an electron-withdrawing group;
R₁ is hydrogen or alkyl_{(C≤12)}, cycloalkyl_{(C≤12)}, or a substituted version of either of these groups;
R₂ is acyl_{(C≤12)} or substituted acyl_{(C≤12)};
R₃ is hydrogen, alkyl_{(C≤12)}, cycloalkyl_{(C≤12)}, substituted alkyl_{(C≤12)} or substituted cycloalkyl_{(C≤12)}; and
R₄ and R₅ are each independently hydrogen, alkyl_{(C≤12)}, cycloalkyl_{(C≤12)}, substituted alkyl_{(C≤12)} or substituted cycloalkyl_{(C≤12)}; or R₄ and R₅ are taken together and are alkanediyl_{(C≤8)} or substituted alkanediyl_{(C≤8)};
or a pharmaceutically acceptable salt, stereoisomer, or tautomer thereof.

3. The compound of claim 1 further defined as: 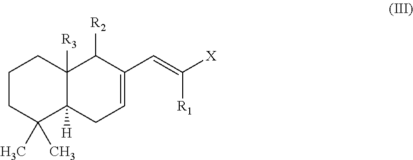
wherein:
X is an electron-withdrawing group;
R₁ is hydrogen or alkyl_{(C≤12)}, cycloalkyl_{(C≤12)}, or a substituted version of either of these groups;
R₂ is acyl_{(C≤12)} or substituted acyl_{(C≤12)}; and
R₃ is hydrogen, alkyl_{(C≤12)}, cycloalkyl_{(C≤12)}, substituted alkyl_{(C≤12)} or substituted cycloalkyl_{(C≤12)};
or a pharmaceutically acceptable salt, stereoisomer, or tautomer thereof.

4. The compound of claim 1 further defined as: 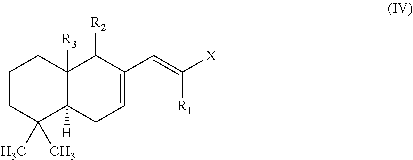
wherein:
X is an electron-withdrawing group;
R₁ is hydrogen or alkyl_{(C≤12)}, cycloalkyl_{(C≤12)}, or a substituted version of either of these groups; and
R₂ is acyl_{(C≤12)} or substituted acyl_{(C≤12)};
or a pharmaceutically acceptable salt, stereoisomer, or tautomer thereof.

5. The compound of claim 1, wherein the electron-withdrawing group is amino, cyano, halo, hydroxy, or nitro.

6. (canceled)

7. The compound of claim 1, wherein the electron-withdrawing group is acyl_{(C≤12)} or substituted acyl_{(C≤12)}.

8. (canceled)

9. The compound of claim 1, wherein the electron-withdrawing group is an alkylphosphonate_{(C≤12)}, dialkylphosphonate_{(C≤12)}, or a substituted version of either of these groups.

10-11. (canceled)

12. The compound of claim 1, wherein the electron-withdrawing group is —Y—C(O)—Z, wherein:
Y is a covalent bond, alkanediyl_{(C≤6)}, alkenediyl_{(C≤6)}, or alkynediyl_{(C≤6)}, or a substituted version of any of these groups; and
Z is hydroxy or alkoxy_{(C≤12)}, aryloxy_{(C≤12)}, aralkoxy_{(C≤12)}, or a substituted version of any of these groups.

13-27. (canceled)

28. A compound of the formula: 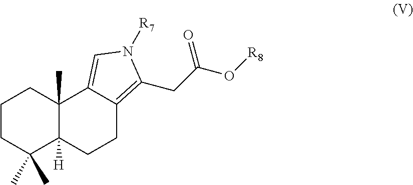
wherein:
R₇ is hydrogen or alkyl_{(C≤12)}, aralkyl_{(C≤12)}, or a substituted version of either of these groups; and
R₈ is hydrogen, alkyl_{(C≤12)}, or substituted alkyl_{(C≤12)};
or a pharmaceutically acceptable salt, stereoisomer, or tautomer thereof.

29-31. (canceled)

32. A pharmaceutical composition comprising:
(a) a compound of claim 1; and
(b) a pharmaceutically acceptable excipient.

33-34. (canceled)

35. A pharmaceutical composition comprising:
(a) polygodial, epi-polygodial, or a stereoisomer thereof; and
(b) a pharmaceutically acceptable excipient;
formulated for administration by injection to a tumor.

36. A method of treating cancer in a patient comprising administering to the patient in need thereof a therapeutically effective amount of a compound or composition of claim 1.

37. The method of claim 36, wherein the cancer is a carcinoma, sarcoma, lymphoma, leukemia, melanoma, mesothelioma, multiple myeloma, or seminoma.

38. The method of claim 36, wherein the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

39-50. (canceled)

51. The method of claim 36, wherein the cancer is resistant to apoptosis.

52-53. (canceled)

54. The method of claim 36, wherein the method comprises injecting the compound directly into the tumor.

55. The method according to claim 36, wherein the method comprises administering the compound systemically.

56. (canceled)

57. The method of claim 36, wherein the method further comprises administering a second therapeutic regimen to said patient.

58-60. (canceled)

61. The method of claim 35, wherein treating comprises reducing the size of a solid tumor in said patient.

62. A method of preparing a compound of formula I comprising reacting a compound of the formula: 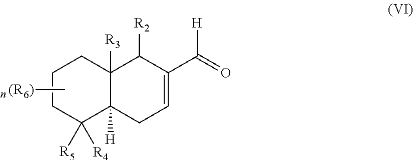
wherein:
R₂ is acyl_{(C≤12)} or substituted acyl_{(C≤12)};
R₃ is hydrogen, alkyl_{(C≤12)}, cycloalkyl_{(C≤12)}, substituted alkyl_{(C≤12)} or substituted cycloalkyl_{(C≤12)};
R₄ and R₅ are each independently hydrogen, alkyl_{(C≤12)}, cycloalkyl_{(C≤12)}, substituted alkyl_{(C≤12)} or substituted cycloalkyl_{(C≤12)}; or R₄ and R₅ are taken together and are alkanediyl_{(C≤8)} or substituted alkanediyl_{(C≤8)};
R₆ is amino, cyano, halo, hydroxy, or nitro;
alkyl_{(C≤6)}, cycloalkyl_{(C≤6)}, acyl_{(C≤6)}, alkoxy_{(C≤6)}, acyloxy_{(C≤6)}, amido_{(C≤6)}, or a substituted version of any of these groups; and
n is 0, 1, 2, 3, or 4;
with a compound of the formula: 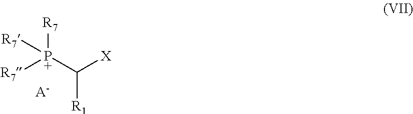
wherein:
A⁻ is a monovalent anion;
X is an electron-withdrawing group;
R₁ is hydrogen or alkyl_{(C≤12)}, cycloalkyl_{(C≤12)}, or a substituted version of either of these groups; and
R₇, R₇′, and R₇″ are each independently aryl_{(C≤12)} or substituted aryl_{(C≤12)};
in the presence of a base.

63. (canceled)
